(19) United States
(12) Patent Application Publication
(10) Pub. No.: US 2013/0123514 A1
(43) Pub. Date: May 16, 2013

(54) CARBOCATALYSTS FOR CHEMICAL TRANSFORMATIONS

(71) Applicants: Christopher W. Bielawski, Austin, TX (US); Daniel R. Dreyer, Austin, TX (US); Hong-Peng Jia, Villeurbanne Cedex (FR)

(72) Inventors: Christopher W. Bielawski, Austin, TX (US); Daniel R. Dreyer, Austin, TX (US); Hong-Peng Jia, Villeurbanne Cedex (FR)

(73) Assignee: GRAPHEA, INC., Austin, TX (US)

(21) Appl. No.: 13/700,430

(22) PCT Filed: May 27, 2011
(86) PCT No.: PCT/US2011/038334

§ 371 Date: Jan. 23, 2013

Related U.S. Application Data
(60) Provisional application No. 61/349,378, filed on May 28, 2010.
(60) Provisional application No. 61/440,574, filed on Feb. 8, 2011.

(51) Int. Cl. C07C 315/02; C07C 45/28; C07C 319/24; C07D 333/22; C07C 45/29; C07C 45/30; C07C 201/12

(52) U.S. Cl. CPC C07C 315/02; C07C 45/30; C07C 45/28; C07C 201/12; C07D 333/22; C07C 45/29; C07C 319/24
USPC 549/72; 252/183.11; 568/323; 568/322; 568/306; 568/361; 549/70; 568/26; 568/22; 568/25; 564/430; 568/23; 568/24; 568/27; 568/37

(57) ABSTRACT

The disclosure relates to catalytically active carbocatalysts, e.g., a graphene oxide or graphite oxide catalyst suitable for use in a variety of chemical transformations. In one embodiment, it relates to a method of catalyzing a chemical reaction of an organic molecule by reacting the organic molecule in the presence of a sufficient amount of graphene oxide or graphite oxide for a time and at a temperature sufficient to allow catalysis of a chemical reaction. According to other embodiments, the reaction may be an oxidation reaction, a hydration reaction, a dehydrogenation reaction, a condensation reaction, or a polymerization reaction. Some reactions may include auto-tandem reactions. The disclosure further provides reaction mixtures containing an organic molecule and graphene oxide or graphite oxide in an amount sufficient to catalyze a reaction of the organic molecule.

### CROSS-REFERENCE

This application claims the benefit of U.S. Provisional Application No. 61/349,378, filed May 28, 2010, and U.S. Provisional Application No. 61/440,574, filed Feb. 8, 2011, which are entirely incorporated herein by reference.

### STATEMENT AS TO FEDERALLY SPONSORED RESEARCH

At least a portion of this invention was developed with the support of the United States government under Grant DMR-0907324 from The National Science Foundation.

### BACKGROUND OF THE INVENTION

Organic material transformations such as redox reactions, hydration reactions, dehydrogenation reactions, condensation reactions and the like are catalyzed by a variety of chemical catalysts. However, currently available catalysts and/or reaction methods have a number of drawbacks, such as expense, toxicity, environmental incompatibility, difficulty in separation from the reaction product, complex reaction conditions, lack of selectivity, lack of compatibility with functional groups, and inefficient catalysis.

For instance, oxidation reactions are used in a wide variety of chemical production methods. Currently, oxidation reactions tend to use a metal catalyst containing either a metal alone or in combination with another chemical. The use of metal catalysts has various drawbacks, such as metal contamination of the resulting products. This is particularly a problem in industries where the product is intended for biological use or other uses sensitive to the presence of metals. Metal catalysts are also often not selective in oxidation reactions and many do not tolerate the presence of functional groups well.

### SUMMARY OF THE INVENTION

Described herein are methods and processes having broad synthetic utility.

Provided herein are methods for chemically transforming an organic compound, comprising:
(a) contacting the organic compound with a catalytically active carbocatalyst; and
(b) transforming the organic compound with the aid of the catalytically active carbocatalyst to form a mixture of a reaction product and a spent or partially spent carbocatalyst.

In one embodiment, the catalytically active carbocatalyst is an oxidized form of graphite. In some of such embodiments, the catalytically active carbocatalyst is graphene oxide or graphite oxide. In some of such embodiments, the catalytically active carbocatalyst is an oxidized carbon-containing material.

In certain embodiments, the catalytically active carbocatalyst is characterized by one or more FT-IR features at about 3150 cm⁻¹,1685 cm⁻¹, 1280 cm⁻¹, or 1140 cm⁻¹.

In a specific embodiment, the catalytically active carbocatalyst is a heterogenous catalyst.

In some embodiments, the catalytically active carbocatalyst provides a reaction solution pH which is neutral upon dispersion in a reaction mixture. In some embodiments, the catalytically active carbocatalyst provides a reaction solution pH which is acidic upon dispersion in a reaction mixture. In some embodiments, the catalytically active carbocatalyst provides a reaction solution pH which is basic upon dispersion in a reaction mixture.

In some embodiments of the methods described above, the transformation is an oxidation. In some embodiments of the methods described above, the transformation is a hydration. In some embodiments of the methods described above, the transformation is a dehydrogenation or aromatization. In some embodiments of the methods described above, the transformation is condensation or a tandem oxidation-condensation (e.g., an aldol reaction or a Claisen-Schmidt reaction).

In some embodiments of the methods described above, the organic compound comprises a secondary alcohol, the transformation is an oxidation and the reaction product is a ketone. In some embodiments of the methods described above, the organic compound comprises a primary alcohol, the transformation is an oxidation and the reaction product is an aldehyde.

In some embodiments of the methods described above, the organic compound comprises an unsubstituted, mono-substituted alkyne, the transformation is a hydration and the reaction product is a ketone.

In some embodiments of the methods described above, the organic compound comprises a di-substituted alkyne, the transformation is a hydration and the reaction product is a ketone.

In some embodiments of the methods described above, the organic compound comprises a nitrile, the transformation is a hydration and the reaction product is a carboxylic acid.

In some embodiments of the methods described above, the organic compound comprises an alcohol or aldehyde, and the transformation is a C—C bond forming condensation with an alkyne or a methyl ketone. In some of such embodiments, the C—C bond formation comprises a part of an auto-tandem oxidation-hydration-aldol coupling reaction.

In some embodiments of the methods described above, the organic compound comprises an olefin, the transformation is an oxidation and the reaction product is a dione.

In some embodiments of the methods described above, the organic compound comprises an olefin, the transformation is a hydration, and the reaction product is an alcohol. In some of such embodiments, the olefin is an unsubstituted, mono-, di-, tri- or tetra-substituted olefin. In some of such embodiments, the olefin is a cis or trans olefin.

In some embodiments of the methods described above, the organic compound comprises an imine, the transformation is an oxidation and the reaction product is an aldehyde, a carboxylic acid or an N-oxide.

In some embodiments of the methods described above, the organic compound is a methyl benzene, the transformation is an oxidation, and the reaction product is a benzaldehyde.

In some embodiments of the methods described above, the organic compound comprises a disubstituted methylene, the transformation is an oxidation and the reaction product is a ketone.

In some embodiments of the methods described above, the organic compound comprises a saturated or partially saturated carbocycle or heterocycle, the transformation is an aromatization or a dehydrogenation, and the reaction product is an aromatic moiety.

In some embodiments of the methods described above, the organic compound comprises a thiol, the transformation is an oxidation, and the reaction product is a disulfide.

In some embodiments of the methods described above, the organic compound comprises a sulfide, the transformation is an oxidation, and the reaction product is a sulfoxide.

In some embodiments of the methods described above, the organic compound comprises a nitrogen containing heterocycle, the transformation is an oxidation, and the reaction product is an N-oxide.

In some embodiments of the methods described above, the organic compound comprises an amine, the transformation is an oxidation, and the reaction product is an N-oxide, a nitroso compound, a nitro compound or a diazo compound.

In some embodiments of the methods described above, the organic compound has at least one sp³-hybridized C—H bond, and the transformation involves the activation of the sp³-hybridized C—H bond by means of the graphene oxide or graphite oxide. In some of such embodiments, the organic compound has at least one sp²-hybridized C—H bond, and the transformation involves the activation of the sp²-hybridized C—H bond by means of the graphene oxide or graphite oxide.

In some embodiments of the methods described above, the graphene oxide or graphite oxide catalyst includes at least 0.0001 wt % graphene oxide or graphite oxide.

In some embodiments of the methods described above, the methods further comprise regenerating the graphene oxide or graphite oxide catalyst after oxidation, hydration or condensation of an organic compound.

In some embodiments of the methods described above, the organic compound is brought in contact with the graphene oxide or graphite oxide catalyst for a time of at least 1 second and/or at a temperature of at least 0° C.

In some embodiments of the methods described above, the graphene oxide or graphite oxide or carbocatalyst has less than 1 part per million transition metal. In some embodiments of the methods described above, the graphene oxide or graphite oxide or carbocatalyst has less than 5 part per billion transition metal. In some embodiments of the methods described above, the graphene oxide or graphite oxide or carbocatalyst has less than 50 part per billion transition metal.

In some embodiments of the methods described above, the reaction product is a pharmaceutical reagent or an intermediate for making a pharmaceutical reagent.

In some embodiments of the methods described above, the reaction product is selected from the group consisting of butyraldehyde, butanoic acid, hexanal, hexanoic acid, benzaldehyde, benzoic acid, cyclohexanone, acetone, acetophenone, benzophenone, naphthalene, benzil, propyl phenyl ketone, benzyl phenyl ketone, 4,4′-diacetylbenzene, methyl octyl ketone, methyl ethyl ketone, formaldehyde, formic acid, carbon dioxide, carbon monoxide, acetaldehyde, ethanoic acid, phenanthrene, benzene, anthracene, anthraquinone, or fluorenone.

In some embodiments the methods described herein further comprise filtration or centrifugation of the mixture of the reaction product and graphene oxide or graphite oxide to produce a reaction product substantially free of graphene oxide or graphite oxide.

In some embodiments of the methods described above, the graphene or graphite oxide has a plurality of functional groups selected from a hydroxyl group, an alkyl group, an alkenyl group, an alkynyl group, an aryl group, epoxide group, peroxide group, peroxyacid group, aldehyde group, ketone group, ether group, carboxylic acid or carboxylate group, peroxide or hydroperoxide group, lactone group, thiolactone, lactam, thiolactam, quinone group, anhydride group, ester group, carbonate group, acetal group, hemiacetal group, ketal group, hemiketal group, aminal, hemiaminal, carbamate, isocyanate, isothiocyanate, cyanamide, hydrazine, hydrazide, carbodiimide, oxime, oxime ether, N-heterocycle, N-oxide, hydroxylamine, hydrazine, semicarbazone, thiosemicarbazone, urea, isourea, thiourea, isothiourea, enamine, enol ether, aliphatic, aromatic, phenolic, thiol, thioether, thioester, dithioester, disulfide, sulfoxide, sulfone, sultone, sulfinic acid, sulfenic acid, sulfenic ester, sulfonic acid, sulfite, sulfate, sulfonate, sulfonamide, sulfonyl halide, thiocyanate, thiol, thial, S-heterocycle, silyl, trimethylsilyl, phosphine, phosphate, phosphoric acid amide, thiophosphate, thiophosphoric acid amide, phosphonate, phosphinite, phosphite, phosphate ester, phosphonate diester, phosphine oxide, amine, imine, amide, aliphatic amide, aromatic amide, halogen, chloro, iodo, fluoro, bromo, acyl halide, acyl fluoride, acyl chloride, acyl bromide, acyl iodide, acyl cyanide, acyl azide, ketene, alpha-beta unsaturated ester, alpha-beta unsaturated ketone, alpha-beta unsaturated aldehyde, anhydride, azide, diazo, diazonium, nitrate, nitrate ester, nitroso, nitrile, nitrite, orthoester group, orthocarbonate ester group, O-heterocycle, borane, boronic acid, boronic ester.

In some embodiments of the methods described above, the transformation is catalytic or stoichiometric with respect to the amount of catalytically active graphene oxide or catalytically active graphite oxide or catalytically active carbocatalyst.

In some embodiments of the methods described above, the organic compound further comprises one or more electron withdrawing groups.

In some embodiments of the methods described above, the organic compound further comprises one or more electron donating groups.

In one aspect, provided herein is a reaction mixture formed according to any methods described above or below.

In one aspect, provided herein is a reaction product according to any of the methods or processes described above or below.

In a further aspect, provided herein is a method for transforming an organic compound having a least one C—H bond, the method comprising:
(a) providing a catalytically active carbocatalyst exhibiting one or more Fourier Transform Infrared (FT-IR) feature at about 3150 cm⁻¹,1685 cm⁻¹, 1280 cm⁻¹, or 1140 cm⁻¹; and
(b) contacting the organic compound with the catalytically active carbocatalyst, thereby transforming the C—H bond of the organic compound, and converting the carbocatalyst to a carbon-containing surface, the carbon-containing surface exhibiting an FT-IR spectrum with one or more attenuated feature at about 3150 cm⁻¹,1685 cm⁻¹, 1280 cm⁻¹, or 1140 cm⁻¹.

In a further aspect is a reaction mixture for use in an oxidation, hydration, dehydrogenation or condensation reaction, comprising:
(a) an organic molecule; and
(b) a catalytically active carbocatalyst for facilitating the oxidation, hydration, dehydrogenation or condensation reaction of the organic molecule, the carbocatalyst having less than 1 part per million metal.

In some embodiments of the reaction mixture, the carbocatalyst exhibits one or more Fourier Transform Infrared (FT-IR) feature at about 3150 cm⁻¹, 1685 cm⁻¹, 1280 cm⁻¹ or 1140 cm⁻¹.

In some embodiments of the reaction mixture, the carbocatalyst has less than 1000 wt % graphene oxide or graphite oxide.

In some embodiments of the reaction mixture, the organic molecule comprises one or more C—H bonds operable to be acted upon by the graphene oxide or graphite oxide or carbocatalyst during the oxidation or polymerization of the organic molecule.

In some embodiments of the reaction mixture, the organic molecule comprises an alkene. In some embodiments of the reaction mixture, the alkene comprises an aromatic alkene. In some embodiments of the reaction mixture, the alkene comprises an aliphatic alkene. In some embodiments of the reaction mixture, the alkene is an unsubstituted, mono-, di-, tri- or tetra-substituted alkene. In some embodiments of the reaction mixture, the alkene is a cis or trans alkene. In some embodiments of the reaction mixture, the organic molecule comprises an alkane.

In some embodiments of the reaction mixture, the carbocatalyst facilitates the oxidation of the alkane to an alcohol, aldehyde or carboxylic acid.

In some embodiments of the reaction mixture, the organic molecule comprises an alkyne. In some embodiments of the reaction mixture, the alkyne is an aliphatic alkyne or aromatic alkyne. In some embodiments of the reaction mixture, the organic molecule comprises a methyl group. In some embodiments of the reaction mixture, the organic molecule comprises disubstituted or trisubstituted methylene. In some embodiments of the reaction mixture, the disubstituted methylene comprises a diarylmethane.

In some embodiments of the reaction mixture, the organic molecule comprises a primary or secondary alcohol. In some embodiments of the reaction mixture, the organic molecule comprises an aromatic alcohol. In some embodiments of the reaction mixture, the organic molecule comprises an aliphatic alcohol. In some embodiments of the reaction mixture, the organic molecule comprises a mono or disubstituted alkyne. In some embodiments of the reaction mixture, the mono or disubstituted alkyne is an aromatic alkyne. In some embodiments of the reaction mixture, the mono or disubstituted alkyne is an aliphatic alkyne.

In some embodiments of the reaction mixture, the reaction mixture is a solvent-free mixture.

In some embodiments of the reaction mixture, the catalyst has less than 1 part per billion metal. In some embodiments of the reaction mixture, the catalyst has less than 5 part per billion metal. In some embodiments of the reaction mixture, the catalyst has less than 50 part per billion metal.

In some embodiments of the reaction mixture, the metal includes one or more transition metals selected from the group consisting of W, Fe, Ta, Ni, Au, Ag, Rh, Ru, Pd, Pt, Ir, Co, Mn, Os, Zr, Zn, Mo, Re, Cu, Cr, V, Ti, Nb.

In some embodiments of the reaction mixture, the organic molecule is a sulfur-containing compound. In some embodiments of the reaction mixture, the organic molecule is a nitrogen-containing compound. In some embodiments of the reaction mixture, the organic molecule is a phosphorus-containing compound. In some embodiments of the reaction mixture, the organic molecule is a halogen-containing compound. In some embodiments of the reaction mixture, the organic molecule is a halogen-containing compound. In some embodiments of the reaction mixture, organic molecule is a silicon-containing compound. In some embodiments of the reaction mixture, the organic molecule is a boron-containing compound. In some embodiments of the reaction mixture, the organic molecule is a boron-containing compound.

In some embodiments of the reaction mixture, the carbocatalyst is dispersed in the reaction mixture. In some embodiments of the reaction mixture, the carbocatalyst is present on a solid support.

In some embodiments of the reaction mixture, the mixture further comprises a co-catalyst. In some embodiments of the reaction mixture, the co-catalyst is an oxidation catalyst. In some embodiments of the reaction mixture, the mixture further comprises an additional oxidizing agent.

In some embodiments of the reaction mixture, the mixture is for use in an oxidation reaction. In some embodiments of the reaction mixture, the mixture is for use in a hydration reaction. In some embodiments of the reaction mixture, the mixture is for use in a dehydrogenation reaction. In some embodiments of the reaction mixture, the mixture is for use in a condensation reaction.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example of one graphene oxide or graphite oxide catalyst that may be used in methods of the disclosure.

FIG. 2 shows an example oxidation of an olefins to a dione, according to an embodiment of the current disclosure.

FIG. 3 shows an example oxidation of cis-stilbene to benzil using graphene oxide or graphite oxide, according to an embodiment of the current disclosure.

FIG. 4 shows an example oxidation of a methyl benzene to an aldehyde, according to an embodiment of the current disclosure.

FIG. 5 shows an example oxidation of a diarylmethane to a ketone, according to an embodiment of the current disclosure.

FIG. 6 shows an example dehydrogenation reaction, according to an embodiment of the current disclosure

FIG. 7 shows an example of an alcohol oxidation catalysis reaction according to an embodiment of the current disclosure.

FIG. 8 shows an example reaction scheme for oxidation of benzyl alcohol using graphene oxide or graphite oxide, according to an embodiment of the current disclosure.

FIG. 9 shows an example reaction scheme for an auto-tandem oxidation-hydration-aldol coupling reaction starting materials, using a specific example, using a graphene oxide or graphite oxide catalyst and phenylacetylene and benzyl alcohol starting materials, according to an embodiment of the current disclosure.

FIG. 10 shows exemplary oxidations of sulfur containing compounds using a graphene oxide or graphite oxide catalyst with thiophenol and phenydisulfide starting materials, according to an embodiment of the current disclosure.

FIG. 11 shows X-ray Photoelectron Spectroscopy (XPS) performed on samples of as-prepared GO.

FIG. 12 shows XPS performed on samples of carbon material recovered after reacting thiophenol with GO (60 wt %) at 100° C. for 10 min.

FIG. 13 shows transmission FT-IR (KBr) of GO (solid line) and the carbon material recovered after reacting thiophenol with GO (60 wt %) at 100° C. for 10 min (dashed line).

### DETAILED DESCRIPTION OF THE INVENTION

### Definitions

The term “catalyst,” as used herein, refers to substance or species that facilitates one or more chemical reactions. A catalyst includes one or more reactive active sites for facilitating a chemical reaction, such as, for example, surface moieties (e.g., OH groups, aldehydes, carboxylic acids). The term catalyst includes a graphene oxide, graphite oxide, or other carbon and oxygen-containing material that facilitates a chemical reaction, such as an oxidation reaction or polymerization reaction. In some situations, the catalyst is incorporated into the reaction product and/or byproduct. As one example, a graphene or graphite oxide catalyst for facilitating a polymerization reaction is at least partially incorporated into a polymer matrix of the polymer formed in the reaction.

The term “carbocatalyst,” as used herein, refers to a catalyst that includes graphite, graphite oxide, graphene, graphene oxide, or closely related carbon materials for the transformation or synthesis of organic or inorganic substrates, or the polymerization of monomeric subunits (also “monomers” herein).

The term “spent catalyst,” as used herein, refers to a catalyst that has been exposed to a reactant to generate a product. In some situations, a spent catalyst is incapable of facilitating a chemical reaction. A spent catalyst has reduced activity with respect to a freshly generated catalyst (also “fresh catalyst” herein). The spent catalyst is partially or wholly deactivated. In some cases, such reduced activity is ascribed to a decrease in the number of reactive active sites.

The term “heterogeneous catalyst,” as used herein, refers to a solid-phase species configured to facilitate a chemical transformation. In heterogeneous catalysis, the phase of the heterogeneous catalyst generally differs from the phase of the reactants(s). A heterogeneous catalyst includes a catalytically active material on a solid support. In some cases the support is catalytically active or inactive. In some situations, the catalytically active material and the solid support is collectively referred to as a “heterogeneous catalyst” (or “catalyst”).

The term “solid support,” as used herein, refers to a support structure for holding or supporting a catalytically active material, such as a catalyst (e.g., carbocatalyst). In some cases, a solid support does not facilitate a chemical reaction. However, in other cases the solid support takes part in a chemical reaction.

The term “nascent catalyst,” as used herein, refers to a substance or material that is used to form a catalyst. A nascent catalyst is characterized as a species that has the potential for acting as a catalyst, such as, upon additional processing or chemical and/or physical modification or transformation.

The term “surface,” as used herein, refers to the boundary between a liquid and a solid, a gas and a solid, a solid and a solid, or a liquid and a gas. A species on a surface has decreased degrees of freedom with respect to the species in the liquid, solid or gas phase.

The term “graphene oxide,” as used herein, refers to catalytically active graphene oxide.

The term “graphite oxide,” as used herein, refers to catalytically active graphite oxide.

“Alcohol” means a —OH radical. In some embodiments an alcohol group is covalently bonded to a surface as described herein.

“Aldehyde” means —C(═O)H radical. In some embodiments an aldehyde group is covalently bonded to a surface as described herein.

“Alkenyl” means a

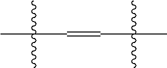 [SMILES: *C=C*]

radical. In some embodiments an alkenyl group is covalently bonded to a surface as described herein.

“Alkynyl” means a

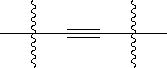 [SMILES: *C#C*]

radical. In some embodiments an alkynyl group is covalently bonded to a surface as described herein.

“Amino” means a —NR′R radical where R and R′ are independently hydrogen, alkyl, aryl, cycloalkyl, heterocycloalkyl or heteroaryl as defined herein, e.g., methylamino, ethylamino, propylamino, aniline, and the like. In some embodiments an amino group is covalently bonded to a surface as described herein.

“Aryl” means a monovalent monocyclic or bicyclic aromatic hydrocarbon radical of 6 to 10 ring atoms e.g., phenyl or naphthyl. In some embodiments an aryl group is covalently bonded to a surface as described herein.

“Cycloalkyl” means a cyclic saturated monovalent hydrocarbon radical of three to ten carbon atoms wherein one or two carbon atoms may be replaced by an oxo group, e.g., cyclopropyl, cyclobutyl, cyclopentyl, or cyclohexyl, and the like. In some embodiments a cycloalkyl group is covalently bonded to a surface as described herein.

“Heterocycloalkyl” means a saturated or unsaturated monovalent monocyclic group of 4 to 8 ring atoms in which one or two ring atoms are heteroatom selected from N, O, or S(O)n, where n is an integer from 0 to 2, the remaining ring atoms being C. The heterocyclyl ring is optionally fused to a (one) aryl or heteroaryl ring as defined herein provided the aryl and heteroaryl rings are monocyclic. The heterocyclyl ring fused to monocyclic aryl or heteroaryl ring is also referred to in this application as “bicyclic heterocyclyl” ring. Additionally, one or two ring carbon atoms in the heterocyclyl ring can optionally be replaced by a —CO— group. More specifically the term heterocyclyl includes, but is not limited to, pyrrolidino, piperidino, homopiperidino, 2-oxopyrrolidinyl, 2-oxopiperidinyl, morpholino, piperazino, tetrahydropyranyl, thiomorpholino, and the like. When the heterocyclyl ring is unsaturated it can contain one or two ring double bonds provided that the ring is not aromatic. When the heterocyclyl group contains at least one nitrogen atom, it is also referred to herein as heterocycloamino and is a subset of the heterocyclyl group. When the heterocyclyl group is a saturated ring and is not fused to aryl or heteroaryl ring as stated above, it is also referred to herein as saturated monocyclic heterocyclyl. In some embodiments a heterocycloalkyl group is covalently bonded to a surface as described herein.

“Heterocycle” includes heteroaryl and heterocycloalkyl cyclic compounds.

“Heteroaryl” means a monovalent monocyclic or bicyclic aromatic radical of 5 to 10 ring atoms where one or more, preferably one, two, or three, ring atoms are heteroatom selected from N, O, or S, the remaining ring atoms being carbon. Representative examples include, but are not limited to, pyrrolyl, thienyl, thiazolyl, imidazolyl, furanyl, indolyl, isoindolyl, oxazolyl, isoxazolyl, benzothiazolyl, benzoxazolyl, quinolinyl, isoquinolinyl, pyridinyl, pyrimidinyl, pyrazinyl, pyridazinyl, triazolyl, tetrazolyl, and the like. In some embodiments a heteroaryl group is covalently bonded to a surface as described herein.

“Imine” means a —C═NR— radical where R is hydrogen, alkyl, aryl, cycloalkyl, heterocycloalkyl or heteroaryl as defined herein. In some embodiments an imine group is covalently bonded to a surface as described herein.

“Ketone” means —C(═O)— radical. In some embodiments an ketone group is covalently bonded to a surface as described herein.

“Methyl” group means a —CH₃ radical. In some embodiments a methyl group is covalently bonded to a surface as described herein.

“Methylene” means a —CH₂-radical. In some embodiments a methylene group is covalently bonded to a surface as described herein.

“Nitrile” means —CN. In some embodiments a nitrile group is covalently bonded to a surface as described herein

“Sulfide” means a —S— radical. In some embodiments a sulfide group is covalently bonded to a surface as described herein.

“Thiol” means a —SH radical. In some embodiments a thiol group is covalently bonded to a surface as described herein.

The term “electron withdrawing group” refers to a chemical substituent that modifies the electrostatic forces acting on a nearby chemical reaction center by withdrawing negative charge from that chemical reaction center. Thus, electron withdrawing groups draw electrons away from a reaction center. Examples include and are not limited to nitro, halo (e.g., fluoro, chloro), haloalkyl (e.g., trifluoromethyl), ketones, esters, aldehydes and the like.

The term “electron donating group” refers to a chemical substituent that modifies the electrostatic forces acting on a nearby chemical reaction center by increasing negative charge at that chemical reaction center. Thus, electron donating groups increase electron density at a reaction center. Examples include and are not limited to alkyl, alkoxy, amino substituents.

Recognized herein are various limitations associated with current commercially-available methods catalyzing chemical reactions. For instance, while transition metal-based catalysts may provide reactions rates that are commercially feasible, the use of metal catalysts has various drawbacks, such as metal contamination of the resulting products. This is particularly problematic in industries where the product is intended for health or biological use, or other uses sensitive to the presence of metals. Another drawback of metal catalysts is that metal catalysts are typically not selective in oxidation reactions and may not tolerate the presence of functional groups in the reactants. As another example to illustrate the drawbacks of metal-based catalysts recognized herein, transition metal-based catalysts may be expensive to manufacture and processes employing such catalysts may have considerable startup and maintenance costs.

Accordingly there is a need for broad-spectrum catalysts that overcome one or more drawbacks of existing catalysts and that are able to catalyze a variety of chemical reactions using a wide range of initial reactants or starting materials.

Described herein are processes for organic transformations involving the use of carbocatalysts that combine the benefits of a metal-free synthesis along with the convenience of heterogenous work up. Advantageously, the versatile carbocatalysts and processes utilizing such carbocatalysts that are described herein are applicable to a variety of organic reactions including and not limited to oxidations, reductions, dehydrogenations, hydrations and/or hydrolysis, sulfur oxidations, nitrogen oxidations, aldol reactions, aromatizations, polymerizations, and the like. Methods of the current disclosure may also have applications in the pharmaceutical industry. For example, chalcones are important precursors for flavonoids and other pharmaceutically important materials and have many uses outside of the pharmaceutical industry. Additionally, the lack of metal in graphene oxide or graphite oxide may allow the use of these methods in reactions metal contamination is a concern, such as reactions to produce pharmaceuticals or agricultural products, or in reactions where it would be detrimental, such as where the product will be subjected to further reactions or used in further applications that are sensitive to metal contamination.

The ability of various carbon-based materials to catalyze the extremely wide number of possible chemical reactions has hitherto not been explored in detail. To date such efforts have relied on exploitation of the relatively high surface areas intrinsic to carbon-based materials to enhance the activity of transition metal based catalysts. For example, metal catalysts have been placed on graphene-based materials to take advantage of the high surface area of such materials and to enhance the activities of the transition metal-based catalysts. However, when metals such as Palladium (Pd) and Platinum (Pt) have been placed on graphene oxide materials to form catalysts, the catalytic activity is attributable to Pt or Pd. In contrast, the carbocatalysts described herein are free of transition metals such as Pt or Pd and the reactions are catalyzed by the carbocatalyst.

The carbocatalysts, and processes involving the use of said carbocatalysts, which are described herein are useful for the synthesis of a large number of industrially and commercially important chemicals that would otherwise be difficult or prohibitively expensive to produce. Additionally, some useful chemical reactions involving organic materials have no available catalysts and are therefore unduly slow or costly. In some embodiments, the carbocatalysts provided herein provide access to such previously intractable chemistries. The broad-spectrum catalysts described herein are able to catalyze a variety of chemical reactions using a variety of initial products (starting materials) and provide a non-toxic alternative to other catalysts and/or reactions. The broad spectrum catalyst and methods of using such catalysts that are provided herein overcome one or more drawbacks of existing catalysts and/or processes.

### Carbocatalysts

In an aspect of the invention, carbon-containing catalysts configured to facilitate a chemical reaction, such as an oxidation reaction or a polymerization reaction, are described. In some embodiments, carbon-containing catalysts are catalytically-active graphene oxide, graphite oxide or other carbon and oxygen-containing catalysts, including heterogeneous catalysts. In some situations, a carbon-containing catalyst is a graphene oxide catalyst or a graphite oxide catalyst.

Methods of Preparing Catalytically Active Carbocatalysts

In one aspect, a carbocatalyst suitable for reactions described herein is an oxidized form of graphite, e.g., a graphene or graphite oxide based catalyst. Graphene or graphite oxide used as a catalyst in the present disclosure is produced using known methods. For example, graphene or graphite oxide is produced by the oxidation of graphite using KMnO₄ and NaNO₃ in concentrated sulfuric acid in concentrated sulfuric acid as described in W. S. Hummer Jr. R. E. Offeman, J. Am. Chem. Soc. 80: 1339 (1958) and A. Lerf, et al. J. Phys Chem. B 102: 4477-4482 (1998), both incorporated in material part by reference herein. Graphene or graphite oxide may also be produced by the oxidation of graphite using NaClO₃ in H₂SO₄ and fuming HNO₃ as described in L. Staudenmaier, Ber. Dtsch. Chem. Ges. 31: 1481-1487 (1898); L. Stuadenmaier, Ber. Dtsch. Chem. Ges. 32:1394-1399 (1899); T. Nakajima, et al. Carbon 44: 537-538 (2006), all incorporated in material part by reference herein. Graphene or graphite oxide may also be prepared by a Brodie reaction.

In some embodiments, a method for forming a catalytically-active graphene oxide or catalytically-active graphite oxide catalyst from a nascent catalyst comprises providing the nascent catalyst to a reaction chamber (or “reaction vessel”), the nascent catalyst comprising graphene or graphite on a solid support. Next, the nascent catalyst is heated in the reaction chamber to an elevated temperature. The nascent catalyst is then contacted with a chemical oxidant.

In some embodiments, the chemical oxidant includes at least one or more materials selected from the group consisting of potassium permanganate, hydrogen peroxide, organic peroxides, peroxy acids, ruthenium-containing species (e.g., tetrapropylammonium perruthenate or other perruthenates), lead-containing species (e.g., lead tetraacetate), chromium-containing species (e.g., chromium oxides or chromic acids), iodine-containing species (e.g., periodates), sulfur-containing oxidants (e.g., potassium peroxymonosulfate), molecular oxygen, ozone, chlorine-containing species (e.g., chlorates or perchlorates or hypochlorites), sodium perborate, nitrogen-containing species (e.g., nitrous oxide or dinitrogen tetraoxide), silver containing species (e.g., silver oxide), osmium containing species (e.g., osmium tetraoxide), 2,2′-dipyridyldisulfide, cerium-containing species (e.g., ammonium cerium nitrate), benzoquinone, Dess Martin periodinane, meta-chloroperbenzoic acid, molybdenum containing species (e.g., molybdenum oxides), N-oxides (e.g., pyridine N-oxide), vanadium-containing species (e.g., vanadium oxides), (2,2,6,6-tetramethylpiperidin-1-yl)oxidanyl (TEMPO), or iron-containing species (e.g., potassium ferricyanide).

In other embodiments, the chemical oxidant is a plasma excited species of an oxygen-containing chemical. In an example, the chemical oxidant includes plasma-excited species of O₂, H₂O₂, NO, NO₂, or other chemical oxidants. In such a case, the nascent catalyst in the reaction chamber is contacted with plasma excited species of the oxygen-containing chemical continuously, such as for a predetermined period of time of at least about 0.1 seconds, or 1 second, or 10 seconds, or 30 seconds, or 1 minute, or 10 minutes, or 30 minutes, or 1 hour, or 2 hours, or 3 hours, or 4 hours, or 5 hours, or 6 hours, or 12 hours, or 1 day, or 2 days, or 3 days, or 4 days, or 5 days, or 6 days, or 1 week, or 2 weeks, or 3 weeks, or 1 month, or 2 months, or 3 months, or 4 months, or 5 months, or 6 months. Alternatively, the nascent catalyst in the reaction chamber is contacted with plasma excites species of the oxygen-containing chemical in pulses, such as pulses having a duration of at least about 0.1 seconds, or 1 second, or 10 seconds, or 30 seconds, or 1 minute, or 10 minutes, or 30 minutes, or 1 hour, or 2 hours, or 3 hours, or 4 hours, or 5 hours, or 6 hours, or 12 hours, or 1 day, or 2 days, or 3 days, or 4 days, or 5 days, or 6 days, or 1 week, or 2 weeks, or 3 weeks, or 1 month, or 2 months, or 3 months, or 4 months, or 5 months, or 6 months. In some situations, the nascent catalyst is exposed to the chemical oxidant for a time period between about 0.1 seconds and 100 days.

In some situations, the nascent catalyst is heated during exposure to the chemical oxidant. In an example, the nascent catalyst is heated at a temperature between about 20° C. and 3000° C., or 20° C. and 2000° C., or about 100° C. and 2000° C.

Alternatively, a method for forming a catalytically-active graphene oxide or catalytically-active graphite oxide catalyst from a nascent catalyst includes providing a nascent catalyst comprising graphene or graphite to a reaction chamber. The reaction chamber has a holder or susceptor for holding one or more nascent catalysts. Next, the nascent catalyst is contacted with one or more acids. In some cases, the one or more acids include sulfuric acid. In some cases, the nascent catalyst is pretreated with potassium persulfate before contacting the nascent catalyst with the one or more acids. Next, the nascent catalyst is contacted with a chemical oxidant. Next, the nascent catalyst is contacted with hydrogen peroxide.

As another alternative, a method for forming a catalytically-active graphene oxide or catalytically-active graphite oxide catalyst from a nascent catalyst includes providing a nascent catalyst comprising graphene or graphite to a reaction chamber. Next, the nascent catalyst is contacted with one or more acids. In some cases, the nascent catalyst is pretreated with potassium persulfate before the nascent catalyst is contacted with the one or more acids. In some cases, the one or more acids include sulfuric acid and nitric acid. The nascent catalyst is then contacted with sodium chlorate, potassium chlorate and/or potassium perchlorate.

In some embodiments, a method for forming a carbocatalyst comprises providing a carbon-containing material in a reaction chamber and contacting the carbon-containing material in the reaction chamber with an oxidizing chemical (also “chemical oxidant” herein) for a predetermined period of time until the carbon-to-oxygen ratio of the carbon-containing material is less than or equal to about 1,000,000 to 1. In some cases, the ratio is determined via elemental analysis, such as XPS. In some embodiments, the time sufficient to achieve such carbon-to-oxygen ratio is at least about 0.1 seconds, or 1 second, or 10 seconds, or 30 seconds, or 1 minute, or 10 minutes, or 30 minutes, or 1 hour, or 2 hours, or 3 hours, or 4 hours, or 5 hours, or 6 hours, or 12 hours, or 1 day, or 2 days, or 3 days, or 4 days, or 5 days, or 6 days, or 1 week, or 2 weeks, or 3 weeks, or 1 month, or 2 months, or 3 months, or 4 months, or 5 months, or 6 months. In some cases, the carbon-containing material is contacted with the chemical oxidant until the carbon-to-oxygen ratio, as determined by elemental analysis, is less than or equal to about 500,000 to 1, or 100,000 to 1, or 50,000 to 1, or 10,000 to 1, or 5,000 to 1, or 1,000 to 1, or 500 to 1, or 100 to 1, or 50 to 1, or 10 to 1, or 5 to 1, or 1 to 1.

As an alternative, a method for forming oxidized and catalytically-active graphite or oxidized and catalytically-active graphene comprises providing graphite or graphene in a reaction chamber and contacting the graphite or graphene with an oxidizing chemical until an infrared spectroscopy spectrum of the graphite or graphene exhibits one or more FT-IR features 3150 cm⁻¹,1685 cm⁻¹, 1280 cm⁻¹, or 1140 cm⁻¹.

In some embodiments, methods for regenerating a spent catalyst, such as a carbocatalyst, include providing the spent catalyst in a reaction chamber or vessel and contacting the spent catalyst with a chemical oxidant. In some cases, the chemical oxidant includes one or more material selected from the group above. In other cases, the chemical oxidant is a plasma excited species of an oxygen-containing chemical. In an example, the chemical oxidant includes plasma-excited species of O₂, H₂O₂, NO, NO₂, or other chemical oxidants. In some embodiments, the spent catalyst is contacted with the chemical oxidant continuously or in pulses, as described above. Contacting the spent catalyst with the chemical oxidant produces a carbocatalyst having a catalytically active material. In an example, contacting a spent catalyst covered with graphene or graphite (or other carbon-containing and oxygen deficient material) forms a layer of catalytically-active graphene oxide or graphite oxide.

Also contemplated with the scope of the present disclosure are other methods of preparation of catalytically active graphene or graphite oxide as described in co-pending PCT International Application Titled “GRAPHENE OXIDE AND GRAPHITE OXIDE CATALYSTS AND SYSTEMS” filed on the same day as the present application, which disclosure is incorporated herein by reference.

An advantage of catalytically active graphene or graphite oxide catalyzed reactions described herein is that the carbocatalyst is heterogeneous, i.e. it does not dissolve in the reaction mixture. Many starting materials, such as alcohols, aldehydes, alkynes, methyl ketones, olefins, methyl benzenes, thiols, and disubstituted methylenes, and their reaction products are soluble in a wide range of organic solvents. In chemical reactions comprising such dissolved starting materials, the graphene or graphite oxide remains as a suspended solid throughout the chemical reaction. In some of the aforementioned methods, the graphene or graphite oxide is removed from the reaction product using simple mechanical methods, such as filtration, centrifugation, sedimentation, or other appropriate mechanical separation techniques, eliminating the need for more complicated techniques such as chromatography or distillation to remove the catalyst.

Following a catalytic reaction, the graphene oxide or graphite oxide is in a different chemical form or in the same chemical form. For example, in one embodiment, reactions described herein result in slow reduction or deoxygenation of the graphene oxide or graphite oxide and loss of functional groups. This altered graphene oxide or graphite oxide remaining after catalysis is put to other uses, or it is regenerated. For example, following the catalytic reaction, the graphene or graphite oxide is in a reduced form. This material is very similar to graphene or graphite and may simply be used for graphene or graphite purposes. For example, reduced graphene oxide is used in energy storage devices or field effect transistors. Alternatively, the reduced graphene or graphite oxide is reoxidized to regenerate the graphene or graphite oxide catalyst. In a further embodiment, following a reaction, graphene or graphite oxide used in the reaction is regenerated in situ and is in the same form as at the start of the reaction. Reoxidation methods are the same as those used to generate the graphene or graphite oxide catalyst originally, such as a Hummers, Staudenmaier, or Brodie oxidation. Thus the carbocatalysts described herein provide an economical alternative to metal based catalysts.

In some embodiments of the invention, carbocatalysts are described that are configured for use with oxidation and/or polymerization reactions. Such carbocatalysts enable reaction rates up to and even exceeding that of transition metal-based catalysts, but reduce, if not eliminate, the contamination issues associated with the use of transition metal-based catalysts.

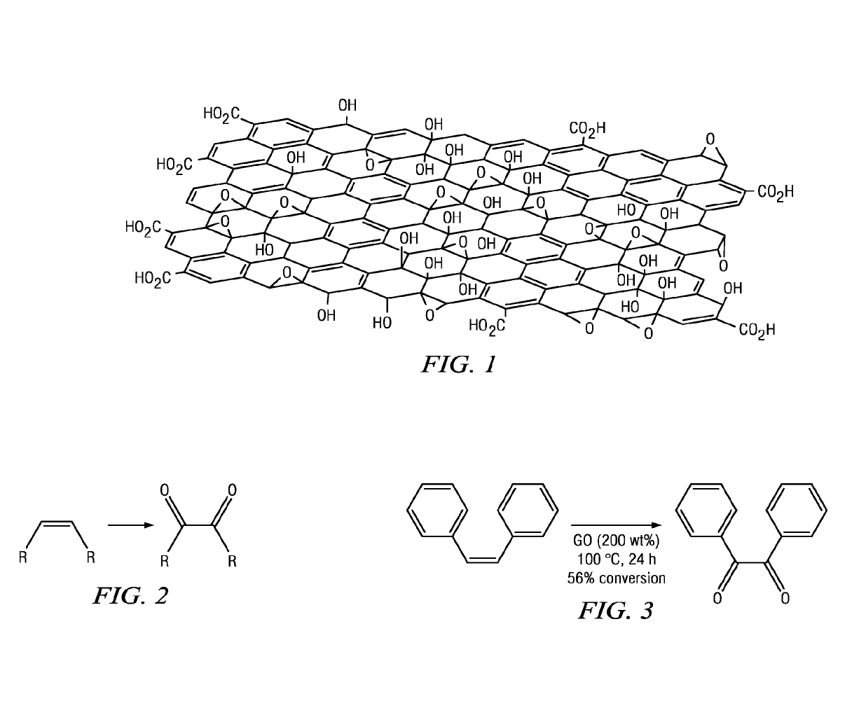

In one embodiment, a carbocatalyst used as a catalyst for any transformation described herein is catalytically active graphene or graphite oxide which comprises one or more oxygen-containing functionalities. An example graphene or graphite oxide catalyst is shown in FIG. 1. In specific embodiments, a graphene of graphite oxide based carbocatalyst described herein contains one or more of alcohols, epoxides, or carboxylic acids. In some situations, at least some of the oxygen-containing functional groups is used to oxidize organic species, such as alkenes and alkynes, or used to polymerize monomeric subunits (also “monomers” herein). In other cases, oxygen is used as a terminal oxidant.

Various embodiments of the invention describe carbocatalysts having graphene oxide at various compositions, concentrations and islands shapes, coverage and adsorption locations.

Also contemplated with the scope of the present disclosure are variations of catalytically active graphene or graphite oxide, including variations in island shapes, coverage and/or adsorption locations, as described in co-pending PCT International Application Titled “GRAPHENE OXIDE AND GRAPHITE OXIDE CATALYSTS AND SYSTEMS” filed on the same day as the present application, which disclosure is incorporated herein by reference.

Carbon-containing catalysts provided herein include unsupported catalytically-active graphene or catalytically-active graphite oxide, as well as graphene or graphite oxide on a solid support, such as a carbon-containing solid support or metal-containing solid support (e.g., TiO₂, Al₂O₃). In alternate embodiments, a solid support is a polymer with a catalytically active graphite oxide or graphene oxide dispersed in the polymer. In some embodiments, catalysts are provided having catalytically-active graphene oxide and/or catalytically-active graphite oxide on a solid support. Examples of such solid supports include carbon nitride, boron nitride, boron-carbon nitride and the like. In other embodiments, catalysts are provided having a catalytically-active carbon and oxygen-containing material and a co-catalyst such as carbon nitride, boron nitride, boron-carbon nitride and the like.

Metal Content

In some situations, catalysts provided herein have a substantially low transition metal content. As an example, a non-transition metal catalyst includes graphene oxide or graphite oxide and have a transition metal content less than or equal to about 1 part per million (ppm). In some cases, the transition metal content of the non-transition metal catalyst is less than or equal to about 0.5 ppm, or less than or equal to about 0.1 ppm, or less than or equal to about 0.06 ppm, or less than or equal to about 0.01 ppm, or less than or equal to about 0.001 ppm, or less than or equal to about 0.0001 ppm, or less than or equal to about 0.00001 ppm. In some situations, the transition metal content of the non-transition metal catalyst is determined by atomic absorption spectroscopy (AAS) or other elemental analysis technique, such as x-ray photoelectron spectroscopy (XPS), or mass spectrometry (e.g., inductively coupled plasma mass spectrometry, or “ICP-MS”).

In some embodiments, the non-transition metal catalyst has a low concentration of transition metals selected from the group consisting of W, Fe, Ta, Ni, Au, Ag, Rh, Ru, Pd, Pt, Ir, Co, Mn, Os, Zr, Zn, Mo, Re, Cu, Cr, V, Ti and Nb. In some embodiments, a non-transition metal catalyst has a metal content (mole %) that is less than about 0.0001%, or less than about 0.000001%, or less than about 0.0000001%.

In some embodiments, a heterogenous catalytically-active graphene oxide or graphite oxide catalyst (or other carbon and oxygen-containing catalyst) is substantially free of metal, particularly transition metal. In some cases, the heterogeneous catalyst has a substantially low metal (e.g., transition metal) concentration of metals selected from the group consisting of W, Fe, Ta, Ni, Au, Ag, Rh, Ru, Pd, Pt, Ir, Co, Mn, Os, Zr, Zn, Mo, Re, Cu, Cr, V, Ti and Nb. In an embodiment, the heterogeneous catalyst has a transition metal concentration that is less than or equal to about 50 part per million, about 20 part per million, about 10 part per million, about 5 part per million, about 1 part per million (“ppm”), or 0.5 ppm, or 0.1 ppm, or 0.06 ppm, or 0.01 ppm, or 0.001 ppm, or 0.0001 ppm, or 0.00001 ppm as measured by atomic absorption spectroscopy or mass spectrometry (e.g., inductively coupled plasma mass spectrometry, or “ICP-MS”). In another embodiment, the heterogeneous catalyst has a metal content (mole %) that is less than about 0.0001%, or less than about 0.000001%, or less than about 0.0000001%.

In some cases, a heterogenous catalytically-active graphene oxide or graphite oxide catalyst (or other carbon and oxygen-containing catalyst) has a substantially low manganese content. In one example the particles have a manganese content that is less than about 1 ppm, or 0.5 ppm, or 0.1 ppm, or 0.06 ppm, or 0.01 ppm, or 0.001 ppm, or 0.0001 ppm, or 0.00001 ppm as measured by atomic absorption spectroscopy or mass spectrometry (e.g., inductively coupled plasma mass spectrometry, or “ICP-MS”).

Surface

In some embodiments, a non-transition metal catalyst having catalytically-active graphene oxide or graphite oxide has a surface that is configured to come in contact with a reactant, such as a hydrocarbon for oxidation or monomeric subunits for polymerization. In some cases, the catalyst has a surface that is terminated by one or more of hydrogen peroxide, hydroxyl groups (OH), aldehyde groups, or carboxylic acid group. In an embodiment, the catalyst has a surface that includes one or more species (or “surface moieties”) selected from the group consisting of hydroxyl group, alkyl group, aryl group, alkenyl group, alkynyl group, epoxide group, peroxide group, peroxyacid group, aldehyde group, ketone group, ether group, carboxylic acid or carboxylate group, peroxide or hydroperoxide group, lactone group, thiolactone, lactam, thiolactam, quinone group, anhydride group, ester group, carbonate group, acetal group, hemiacetal group, ketal group, hemiketal group, aminal, hemiaminal, carbamate, isocyanate, isothiocyanate, cyanamide, hydrazine, hydrazide, carbodiimide, oxime, oxime ether, N-heterocycle, N-oxide, hydroxylamine, hydrazine, semicarbazone, thiosemicarbazone, urea, isourea, thiourea, isothiourea, enamine, enol ether, aliphatic, aromatic, phenolic, thiol, thioether, thioester, dithioester, disulfide, sulfoxide, sulfone, sultone, sulfinic acid, sulfenic acid, sulfenic ester, sulfonic acid, sulfite, sulfate, sulfonate, sulfonamide, sulfonyl halide, thiocyanate, thiol, thial, S-heterocycle, silyl, trimethylsilyl, phosphine, phosphate, phosphoric acid amide, thiophosphate, thiophosphoric acid amide, phosphonate, phosphinite, phosphite, phosphate ester, phosphonate diester, phosphine oxide, amine, imine, amide, aliphatic amide, aromatic amide, halogen, chloro, iodo, fluoro, bromo, acyl halide, acyl fluoride, acyl chloride, acyl bromide, acyl iodide, acyl cyanide, acyl azide, ketene, alpha-beta unsaturated ester, alpha-beta unsaturated ketone, alpha-beta unsaturated aldehyde, anhydride, azide, diazo, diazonium, nitrate, nitrate ester, nitroso, nitrile, nitrite, orthoester group, orthocarbonate ester group, O-heterocycle, borane, boronic acid and boronic ester. In an example, such surface moieties are disposed on the surface at various reactive active sites of the catalyst.

Carbon Content

In some embodiments, a catalytically-active graphene oxide or graphite oxide catalyst (or other carbon and oxygen-containing catalyst) has a carbon content (mole %) of at least about 25%, or 30%, or 35%, or 40%, or 45%, or 50%, or 55%, or 60%, or 65%, or 70%, or 75%, or 80%, or 85%, or 90%, or 95%, or 99%, or 99.99%. The balance of the catalyst is oxygen, or one or more other surface moieties described herein, or one or more elements selected from the group consisting of oxygen, boron, nitrogen, sulfur, phosphorous, fluorine, chlorine, bromine and iodine. In some embodiments, a graphene oxide or graphite oxide has an oxygen content of at least about 0.01%, or 1%, or 5%, or 15%, or 20%, or 25%, or 30%, or 35%, or 40%, or 45%, or 50%. For example, a graphene or graphite oxide catalyst has a carbon content of at least about 25% and an oxygen content of at least about 0.01%. The oxygen content is measured with the aid of various surface or bulk analytical spectroscopic techniques. As one example, the oxygen content is measured by x-ray photoelectron spectroscopy (XPS) or mass spectrometry (e.g., inductively coupled plasma mass spectrometry, or “ICP-MS”).

In some embodiments, a carbocatalyst has a bulk carbon-to-oxygen ratio of at least about 0.1:1, or 0.5:1, or 1:1, or 1.5:1, or 2:1, or 2.5:1, or 3:1, or 3.5:1, or 4:1, or 4.5:1, or 5:1, or 5.5:1, or 6:1, or 6.5:1, or 7:1, or 7.5:1, or 8:1, or 8.5:1, or 9:1, or 9.5:1, or 10:1, or 100:1, or 1000:1, or 10,000:1, or 100,000:1, or 1,000,000:1. In some cases, a carbocatalyst has a surface carbon-to-oxygen ratio of at least about 0.1:1, or 0.5:1, or 1:1, or 1.5:1, or 2:1, or 2.5:1, or 3:1, or 3.5:1, or 4:1, or 4.5:1, or 5:1, or 5.5:1, or 6:1, or 6.5:1, or 7:1, or 7.5:1, or 8:1, or 8.5:1, or 9:1, or 9.5:1, or 10:1, or 100:1, or 1000:1, or 10,000:1, or 100,000:1, or 1,000,000:1.

In some embodiments, a catalytically-active graphene oxide or graphite oxide-containing catalyst has graphene oxide or graphite oxide with a bulk carbon-to-oxygen ratio of at least about 0.1:1, or 0.5:1, or 1:1, or 1.5:1, or 2:1, or 2.5:1, or 3:1, or 3.5:1, or 4:1, or 4.5:1, or 5:1, or 5.5:1, or 6:1, or 6.5:1, or 7:1, or 7.5:1, or 8:1, or 8.5:1, or 9:1, or 9.5:1, or 10:1, or 100:1, or 1000:1, or 10,000:1, or 100,000:1, or 1,000,000:1. In some cases, a graphene oxide or graphite oxide-containing catalyst includes graphene oxide or graphite oxide with a surface carbon-to-oxygen ratio of at least about 0.1:1, or 0.5:1, or 1:1, or 1.5:1, or 2:1, or 2.5:1, or 3:1, or 3.5:1, or 4:1, or 4.5:1, or 5:1, or 5.5:1, or 6:1, or 6.5:1, or 7:1, or 7.5:1, or 8:1, or 8.5:1, or 9:1, or 9.5:1, or 10:1, or 100:1, or 1000:1, or 10,000:1, or 100,000:1, or 1,000,000:1.

pH

In some cases, a heterogenous catalytically active carbocatalyst (e.g., graphene oxide or graphite oxide catalyst, or other carbon and oxygen-containing catalyst) provides a solution pH of between about 0.1 to about 14 when dispersed in solution. In some cases, a heterogenous catalytically active carbocatalyst (e.g., graphene oxide or graphite oxide catalyst, or other carbon and oxygen-containing catalyst) provides a reaction solution pH which is acidic (e.g., pH of between about 0.1 to about 6.9) when dispersed in solution. In some cases, a heterogenous catalytically active carbocatalyst (e.g., graphene oxide or graphite oxide catalyst, or other carbon and oxygen-containing catalyst) provides a reaction solution pH which is basic (e.g., pH of between about 7.1 to about 14) when dispersed in solution. In some cases, a heterogenous catalytically active carbocatalyst (e.g., graphene oxide or graphite oxide catalyst, or other carbon and oxygen-containing catalyst) provides a reaction solution pH which is neutral (e.g., pH of about 7) when dispersed in solution.

By way of example, in one embodiment, “acidic GO” that provides a solution pH of 1-3 versus a solution pH of 4-6 is prepared by eliminating the certain optional steps in the material's preparation that involve washing with water. Normally, after the synthesis of a GO catalyst is performed in acid, the GO is washed with a large volume of water to remove this acid. When the number of wash steps is reduced, a GO catalyst with a large amount of exogenous acid adsorbed to its surface is formed and the pH of the solution is lower compared to the pH when the catalyst is prepared by washing the material with water.

In another embodiment, GO is basified by exposure to a base. Such a basic GO catalyst is prepared by stirring a dispersion of GO in water with non-nucleophilic bases such as potassium carbonate or sodium bicarbonate, and isolated the resulting product by filtration. Such carbocatalysts display significantly higher pH values when dispersed in water (pH=6-8).

Accordingly, depending on choice of substrates (e.g., whether a starting material is sensitive to acid or base) a suitable carbocatalyst is prepared that provides either an acidic or basic pH upon dispersion in solution.

Stoichiometry and Catalyst Loading

In some embodiments, for any catalytically active heterogenous carbocatalyst (e.g., graphene or graphite oxide) mediated reaction described herein, e.g., oxidation, hydration, dehydrogenation/aromatization, polymerization, condensation or tandem oxidation-condensation reactions, the amount of graphene oxide or graphite oxide used is anywhere between 0.01 wt % and 1000 wt %. As used herein, wt % designates weight of the catalyst as compared to the weight of the reactant or reactants. In particular embodiments, the graphene oxide or graphite oxide catalyst may constitute at least 0.01 wt %, between 0.01 wt % and 5 wt %, between 5 wt % and 50 wt %, between 50 wt % and 200 wt %, between 200 wt % and 400 wt %, between 400 wt % and 1000 wt %, or up to 1000 wt %. The amount of catalyst used may vary depending on the type of reaction. For example reactions in which the catalyst acts on a C—H bond may work well at higher amounts of catalyst, such as up to 400 wt %. Other reactions, such a polymerization reactions, may work well at lower catalyst levels, such as as little as 0.01 wt %.

In some situations, the groups present at the surface of a catalytically activated carbocatalyst (e.g., a peroxide moiety covalently bound to graphene or graphite oxide) are modified to provide stoichiometric control of a reaction.

Reaction Time

In some embodiments, for any catalytically active heterogenous carbocatalyst (e.g., graphene or graphite oxide) mediated reaction described herein, e.g., oxidation, hydration, dehydrogenation/aromatization, polymerization, condensation or tandem oxidation-condensation reactions, the duration of the reaction (e.g., for more than about 60%, about 70%, about 80%, about 90%, about 95% or about 100% conversion of starting material to product) is from seconds to minutes, from minutes to hours, or from hours to days. In one embodiment, for any catalytically active heterogenous carbocatalyst mediated reaction described herein, the duration of the reaction is from about 1 second to about 5 minutes. In one embodiment, for any catalytically active heterogenous carbocatalyst mediated reaction described herein, the duration of the reaction is from about 5 minutes to about 30 minutes. In one embodiment, for any catalytically active heterogenous carbocatalyst mediated reaction described herein, the duration of the reaction is from about 30 minutes to about 60 minutes. In one embodiment, for any catalytically active heterogenous carbocatalyst mediated reaction described herein, the duration of the reaction is from about 60 minutes to about 4 hours. In one embodiment, for any catalytically active heterogenous carbocatalyst mediated reaction described herein, the duration of the reaction is from about 4 hours to about 8 hours. In one embodiment, for any catalytically active heterogenous carbocatalyst mediated reaction described herein, the duration of the reaction is from about 8 hours to about 12 hours. In one embodiment, for any catalytically active heterogenous carbocatalyst mediated reaction described herein, the duration of the reaction is from about 8 hours to about 24 hours. In one embodiment, for any catalytically active heterogenous carbocatalyst mediated reaction described herein, the duration of the reaction is from about 24 hours to about 2 days. In one embodiment, for any catalytically active heterogenous carbocatalyst mediated reaction described herein, the duration of the reaction is from about 1 day to about 3 days. In one embodiment, for any catalytically active heterogenous carbocatalyst mediated reaction described herein, the duration of the reaction is from about 1 day to about 5 days. In one embodiment, for any catalytically active heterogenous carbocatalyst mediated reaction described herein, the duration of the reaction is from about 1 day to about 6 days.

Reaction Temperature

In some embodiments, for any catalytically active heterogenous carbocatalyst mediated reaction described herein, an oxidation reaction is carried out at a temperature between about 0° C. and 500° C. In some embodiments, for any catalytically active heterogenous carbocatalyst mediated reaction described herein, an oxidation reaction is carried out at a temperature between about 0° C. and 300° C. In some embodiments, for any catalytically active heterogenous carbocatalyst mediated reaction described herein, an oxidation reaction is carried out at a temperature between about 25° C. and 300° C. In some embodiments, for any catalytically active heterogenous carbocatalyst mediated reaction described herein, an oxidation reaction is carried out at a temperature between about 25° C. and 200° C. In some embodiments, for any catalytically active heterogenous carbocatalyst mediated reaction described herein, an oxidation reaction is carried out at a temperature between about 25° C. and 100° C. In some embodiments, for any catalytically active heterogenous carbocatalyst mediated reaction described herein, an oxidation reaction is carried out at a temperature between about 50° C. and 300° C. IIn some embodiments, for any catalytically active heterogenous carbocatalyst mediated reaction described herein, an oxidation reaction is carried out at a temperature between about 50° C. and 200° C. In some embodiments, for any catalytically active heterogenous carbocatalyst mediated reaction described herein, an oxidation reaction is carried out at a temperature between about 50° C. and 150° C. In some embodiments, for any catalytically active heterogenous carbocatalyst mediated reaction described herein, an oxidation reaction is carried out at a temperature between about 50° C. and 100° C. In some embodiments, for any catalytically active heterogenous carbocatalyst mediated reaction described herein, an oxidation reaction is carried out at a temperature between about 75° C. and 300° C. In some embodiments, for any catalytically active heterogenous carbocatalyst mediated reaction described herein, an oxidation reaction is carried out at a temperature between about 75° C. and 200° C.

In some embodiments, for any catalytically active heterogenous carbocatalyst mediated reaction described herein, a hydration reaction is carried out at a temperature between about 0° C. and 500° C. In some embodiments, for any catalytically active heterogenous carbocatalyst mediated reaction described herein, a hydration reaction is carried out at a temperature between about 0° C. and 300° C. In some embodiments, for any catalytically active heterogenous carbocatalyst mediated reaction described herein, a hydration reaction is carried out at a temperature between about 0° C. and 200° C. In some embodiments, for any catalytically active heterogenous carbocatalyst mediated reaction described herein, a hydration reaction is carried out at a temperature between about 0° C. and 100° C. In some embodiments, for any catalytically active heterogenous carbocatalyst mediated reaction described herein, a hydration reaction is carried out at a temperature between about 25° C. and 300° C. In some embodiments, for any catalytically active heterogenous carbocatalyst mediated reaction described herein, a hydration reaction is carried out at a temperature between about 25° C. and 200° C. In some embodiments, for any catalytically active heterogenous carbocatalyst mediated reaction described herein, a hydration reaction is carried out at a temperature between about 25° C. and 100° C. In some embodiments, for any catalytically active heterogenous carbocatalyst mediated reaction described herein, a hydration reaction is carried out at a temperature between about 50° C. and 300° C. In some embodiments, for any catalytically active heterogenous carbocatalyst mediated reaction described herein, a hydration reaction is carried out at a temperature between about 50° C. and 200° C. In some embodiments, for any catalytically active heterogenous carbocatalyst mediated reaction described herein, a hydration reaction is carried out at a temperature between about 50° C. and 150° C. In some embodiments, for any catalytically active heterogenous carbocatalyst mediated reaction described herein, a hydration reaction is carried out at a temperature between about 50° C. and 100° C. In some embodiments, for any catalytically active heterogenous carbocatalyst mediated reaction described herein, a hydration reaction is carried out at a temperature between about 75° C. and 300° C. In some embodiments, for any catalytically active heterogenous carbocatalyst mediated reaction described herein, a hydration reaction is carried out at a temperature between about 75° C. and 200° C.

In some embodiments, for any catalytically active heterogenous carbocatalyst mediated reaction described herein, a condensation or tandem oxidation-condensation reaction is carried out at a temperature between about 0° C. and 500° C. In some embodiments, for any catalytically active heterogenous carbocatalyst mediated reaction described herein, a condensation or tandem oxidation-condensation reaction is carried out at a temperature between about 0° C. and 300° C. In some embodiments, for any catalytically active heterogenous carbocatalyst mediated reaction described herein, a condensation or tandem oxidation-condensation reaction is carried out at a temperature between about 0° C. and 100° C. In some embodiments, for any catalytically active heterogenous carbocatalyst mediated reaction described herein, a condensation or tandem oxidation-condensation reaction is carried out at a temperature between about 25° C. and 300° C. In some embodiments, for any catalytically active heterogenous carbocatalyst mediated reaction described herein, a condensation or tandem oxidation-condensation reaction is carried out at a temperature between about 25° C. and 200° C. In some embodiments, for any catalytically active heterogenous carbocatalyst mediated reaction described herein, a condensation or tandem oxidation-condensation reaction is carried out at a temperature between about 25° C. and 100° C. In some embodiments, for any catalytically active heterogenous carbocatalyst mediated reaction described herein, a condensation or tandem oxidation-condensation reaction is carried out at a temperature between about 50° C. and 300° C. In some embodiments, for any catalytically active heterogenous carbocatalyst mediated reaction described herein, a condensation or tandem oxidation-condensation reaction is carried out at a temperature between about 50° C. and 200° C. In some embodiments, for any catalytically active heterogenous carbocatalyst mediated reaction described herein, a condensation or tandem oxidation-condensation reaction is carried out at a temperature between about 50° C. and 150° C. In some embodiments, for any catalytically active heterogenous carbocatalyst mediated reaction described herein, a condensation or tandem oxidation-condensation reaction is carried out at a temperature between about 50° C. and 100° C. In some embodiments, for any catalytically active heterogenous carbocatalyst mediated reaction described herein, a condensation or tandem oxidation-condensation reaction is carried out at a temperature between about 75° C. and 300° C. In some embodiments, for any catalytically active heterogenous carbocatalyst mediated reaction described herein, a condensation or tandem oxidation-condensation reaction is carried out at a temperature between about 75° C. and 200° C.

In some embodiments, for any catalytically active heterogenous carbocatalyst mediated reaction described herein, a dehydrogenation/aromatization reaction is carried out at a temperature between about 0° C. and 500° C. In some embodiments, for any catalytically active heterogenous carbocatalyst mediated reaction described herein, a dehydrogenation/aromatization reaction is carried out at a temperature between about 0° C. and 300° C. In some embodiments, for any catalytically active heterogenous carbocatalyst mediated reaction described herein, dehydrogenation/aromatization reaction is carried out at a temperature between about 0° C. and 100° C. In some embodiments, for any catalytically active heterogenous carbocatalyst mediated reaction described herein, dehydrogenation/aromatization reaction is carried out at a temperature between about 25° C. and 300° C. In some embodiments, for any catalytically active heterogenous carbocatalyst mediated reaction described herein, dehydrogenation/aromatization reaction is carried out at a temperature between about 25° C. and 200° C. In some embodiments, for any catalytically active heterogenous carbocatalyst mediated reaction described herein, dehydrogenation/aromatization reaction is carried out at a temperature between about 25° C. and 100° C. In some embodiments, for any catalytically active heterogenous carbocatalyst mediated reaction described herein, dehydrogenation/aromatization reaction is carried out at a temperature between about 50° C. and 300° C. In some embodiments, for any catalytically active heterogenous carbocatalyst mediated reaction described herein, dehydrogenation/aromatization reaction is carried out at a temperature between about 50° C. and 200° C. In some embodiments, for any catalytically active heterogenous carbocatalyst mediated reaction described herein, dehydrogenation/aromatization reaction is carried out at a temperature between about 50° C. and 150° C. In some embodiments, for any catalytically active heterogenous carbocatalyst mediated reaction described herein, dehydrogenation/aromatization reaction is carried out at a temperature between about 50° C. and 100° C. In some embodiments, for any catalytically active heterogenous carbocatalyst mediated reaction described herein, dehydrogenation/aromatization reaction is carried out at a temperature between about 75° C. and 300° C. In some embodiments, for any catalytically active heterogenous carbocatalyst mediated reaction described herein, dehydrogenation/aromatization reaction is carried out at a temperature between about 75° C. and 200° C.

Solvent

In some embodiments, for any catalytically active heterogenous carbocatalyst mediated reaction described herein, a suitable solvent is any solvent having low reactivity toward the carbocatalyst. In one embodiment, a chlorinated solvent is used, e.g., dichloromethane, chloroform, tetrachloromethane, dichloroethane and the like. In other situations, solvents such as acetonitrile or DMF are used. In some embodiments, water is used as a solvent. Less preferred solvents include solvents such as methanol, ethanol and/or tetrahydrofuran.

In further optional embodiments, the reaction is free of solvent. For example, in one case a reaction comprises a gaseous reactant (e.g., methane) which is contacted with a heated catalytically active carbocatalyst as described herein. In another case, a reaction comprises a liquid reactant which is contacted with a catalytically active carbocatalyst as described herein, and the reaction is thereby free of additional solvent. In another case, a reaction comprises a solid reactant which is contacted with a catalytically active carbocatalyst as described herein, wherein upon heating, the solid melts to form a liquid reactant.

Functional Groups

In some embodiments, for any catalytically active heterogenous carbocatalyst mediated reaction described herein, a starting material comprises one or more functional groups. Within such substrates, in one embodiment, only one functional group is transformed (e.g., a substrate comprises an alkene and an alcohol and only the alcohol is oxidized). In an alternate embodiment, more than one functional group is transformed (e.g., an alcohol group is oxidized and a nitrile group is hydrated). In further embodiments, other functional groups present in an organic molecule are not affected by the reaction conditions described herein (i.e., the functional groups are stable to the reaction conditions). For example, a silyl ether is not cleaved under reaction conditions described herein while allowing for conversion of a nitrile to a carboxylic acid.

In further embodiments, a functional group that is transformed is optionally allowed to undergo more than one transformation. For example, a methyl group is transformed to an alcohol and further oxidized to an aldehyde.

Turnover

In some embodiments, for any catalytically active heterogenous carbocatalyst mediated reaction described herein, the turnover number for the reaction is on the order of 10⁻⁵ to about 1,000,000. In some embodiments, for any catalytically active heterogenous carbocatalyst mediated reaction described herein, the turnover number for the reaction is on the order of 10⁻⁴ to about 10⁴. In an exemplary embodiment, for any catalytically active heterogenous carbocatalyst mediated reaction described herein, the turnover number for the reaction is on the order of 10⁻² (expressed in moles of product per mass of catalyst).

Co-Catalyst

In some embodiments, for any catalytically active heterogenous carbocatalyst mediated reaction described herein, the reaction mixture further comprises a co-catalyst. In one embodiment, such a co-catalyst is, for example, carbon nitride, boron nitride, boron carbon nitride, and the like. In some embodiments, a co-catalyst is an oxidation catalyst (e.g., titanium dioxide, Manganese dioxide). In some embodiments, a co-catalyst is a dehydrogenation catalyst (e.g, Pd/ZnO).

Co-Reagents

In further optional embodiments, any carbocatalyst mediated reaction described herein is optionally carried out in the presence of co-reagents. In one embodiment, such a co-reagent is an additional oxidizing reagent such as ozone, hydrogen peroxide, oxone, molecular oxygen, or the like. In another embodiment, an additional reagent may be a complementary reagent having synergy with the procedures described herein such as a Dess Martin periodinane reagent or a Swern oxidation reagent.

Carbocatalyst Catalyzed Transformations

Graphene oxide or graphite oxide is used in a variety of reactions, and is used for oxidation of unactivated substrates (e.g., hydrocarbons) and/or oxidation or hydrations of other reactive substrates (e.g., alknenes, alkynes or other substrates described herein), and/or for condensation or dehydrogenation reactions of a variety of inert or activated substrates. In these reactions, graphene oxide or graphite oxide exerts its catalytic effect through one or more of exemplary properties such as acidic properties, dehydrative properties, oxidative properties, dehydrogenation properties, redox properties, or any combination thereof.

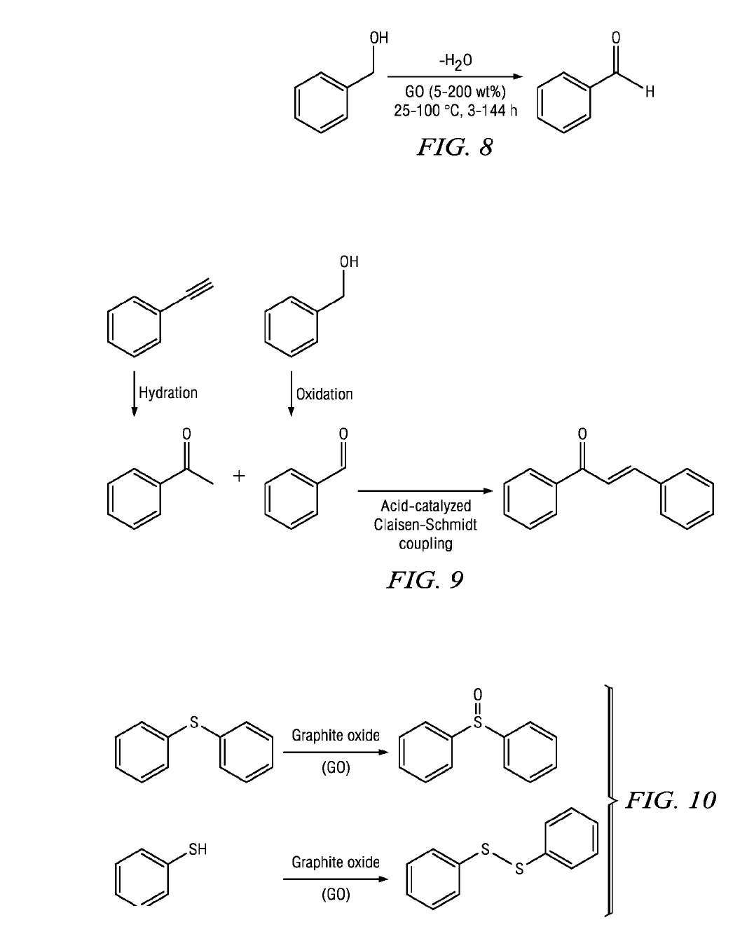

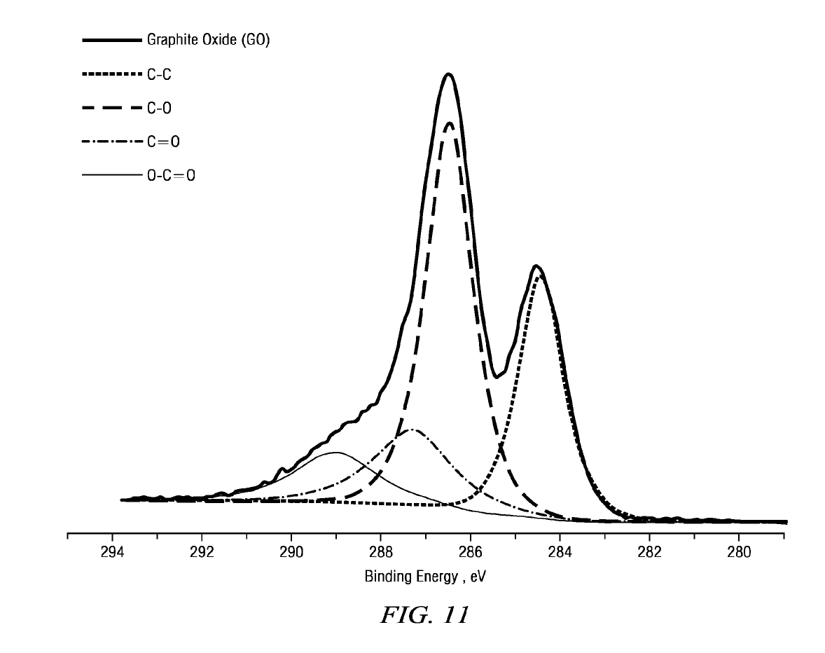

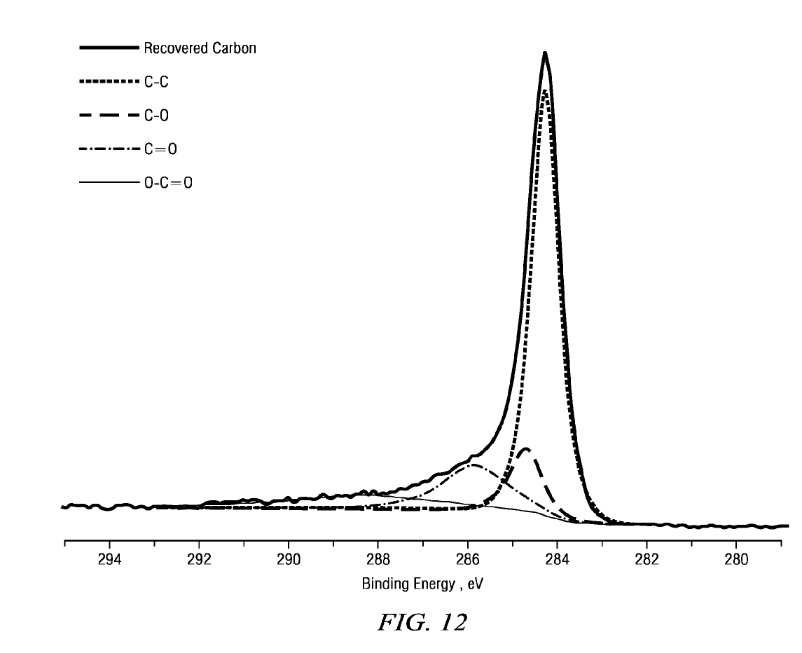

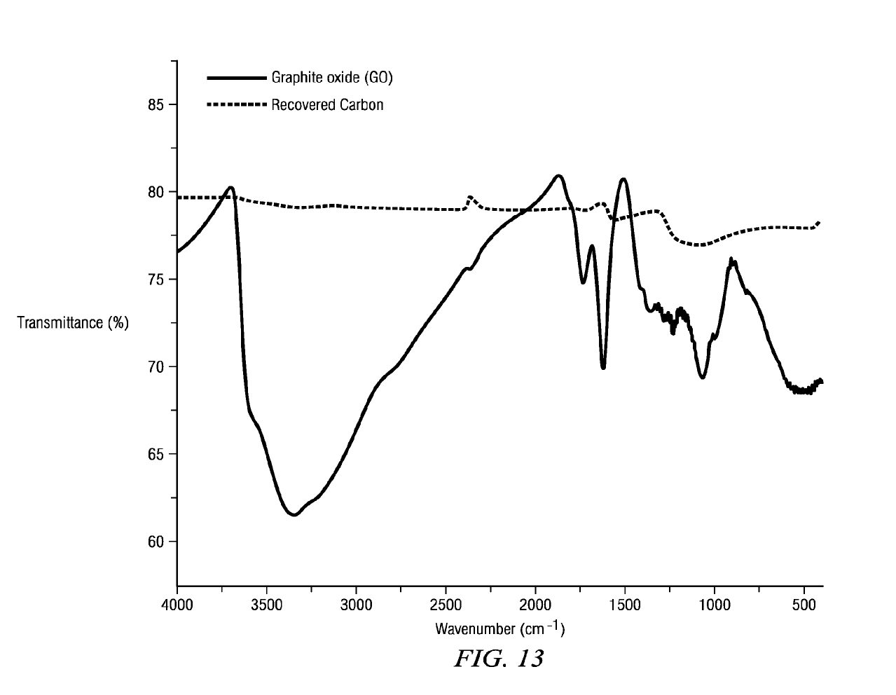

Examples of these reactions are shown in FIGS. 2-6. Further, graphene oxide or graphite oxide can be used for alcohol oxidation or for alkyne hydration as shown in FIGS. 7-8. Graphene oxide or graphite oxide is used to catalyze the formation of a chalcone through a condensation reaction of ketones and aldehydes. Examples of this reaction are shown in FIG. 9. As also shown in FIG. 9, graphene oxide or graphite oxide is used for an auto-tandem catalysis in which alcohol and alkyne starting materials are oxidized and hydrated, respectively, to form ketones and aldehydes the condensation of which into chalcones is also catalyzed by the graphene oxide or graphite oxide. In the auto-tandem catalysis, the reactions take place in the same vessel at essentially the same time and are catalyzed by the addition of graphene oxide or graphite oxide only once at the beginning of the reaction. FIG. 10 shows oxidations of sulfur containing compounds.

### Oxidation Reactions

Graphene oxide or graphite oxide catalyzes a variety of oxidation reactions in which the catalyst acts on one or more of a C—H bond, a sulfur atom, and/or a nitrogen atom in an organic reactant. Example reactions include the oxidation of hydrocarbons, oxidation of alkenes to diones, oxidation of methyl benzenes to their respective aldehydes, oxidation of disubstituted methylenes, such as diarylmethanes, to their respective ketones, and dehydrogenation reactions, such as the aromatization of cyclic, hydrogenated species, for example the aromatization of tetralin to form naphthalene.

### Oxidation of Olefins to Diones

As shown in FIG. 2, graphene oxide or graphite oxide is used to catalyze the oxidation of alkenes (olefins) to diones. In alkene oxidation, the graphene oxide or graphite oxide functions as a catalytic oxidant for the transformation of an alkene to a dione. The catalysis reaction is carried out by mixing graphene oxide or graphite oxide with the alkene under conditions suitable to allow oxidation of the alkene. The diones formed are removed during the reaction (e.g., by fractional distillation) or afterwards.

In specific embodiments, the alkene is aromatic or aliphatic. In specific embodiments, the alkene is unsubstituted (e.g., ethane gas), mono-, di- tri- or tetra-substituted alkene. In some embodiments, the alkene is a cis alkene. In other embodiments, the alkene is a trans alkene. In further embodiments, the alkene is heteroaromatic, a cycloalkene, or a heterocycloalkene. Example alkenes include, and are not limited to, cis-stilbene (FIG. 3) and functionalized derivatives thereof.

In additional embodiments, alkene groups present on saturated, partially unsaturated and/or unsaturated chains or rings such as fatty acids, cycloalkyl, heterocycloalkyl, or heteroaryl rings are also oxidized to the corresponding diones. Other heterocycle-containing substrates and/or other cycloalkyl-containing substrates are expected to show similar reactivity. Examples of such substrates include: heteroaryl ethenes where the heteroaryl rings include imidazole, pyrazole, pyrrole, pyrrolidine, pyridine, piperidine, oxazole, thiazole, triazole (all isomers), tetrazole, thiophene, furan, thiofuran, indole, indoline, and others; cycloalkyl-ethenes where the cylcloalkyls include cyclobutane, cyclopropane, cyclopentane, cyclohexane, cycloheptane, and others; heterocycloalkyl-ethenes where the heterocycloalkyls include pyrrolidines, piperidines, and the like.

In a further aspect, for any of such alkene substrates, perturbation of the electronic properties of substrate alkene (e.g., through the incorporation of electron-donating or -withdrawing substituents does not significantly affect the isolated yield of the desired product. Thus substrates comprising either electron donating groups (e.g., methoxy, dialkylamino, or any other electron donating group) or electron withdrawing groups (e.g., nitro, halo, or any other electron withdrawing group) are amenable to oxidation in the presence of carbocatalysts described herein. Thus the methods of oxidation in the presence of catalytically active carbocatalysts presented herein have wide synthetic utility.

In some exemplary embodiments, reaction is carried out at a temperature of between 100° C. and 120° C. for a duration of 6-48 hours, more particularly 24 hours in the presence of 1-400 wt % graphene oxide or graphite oxide, more particularly 200 wt %.

It will be recognized that any combination of catalyst loadings, reaction times, solvent and reaction temperatures, as described in the sections above, are contemplated as within the scope of any of the embodiments described above or below.

In alkenes or other compounds with large substituents, the substituents and geometry play a role in whether graphene oxide or graphite oxide catalyzes oxidation. For example, the aromatic groups and cis configuration of alkenes affect the suitable or optimal conditions for their oxidation. The role of substituents or geometry stems from steric constraints or the orientation of the interaction between the alkene and the surface of the catalyst.

### Oxidation of Methyl Group to Aldehyde

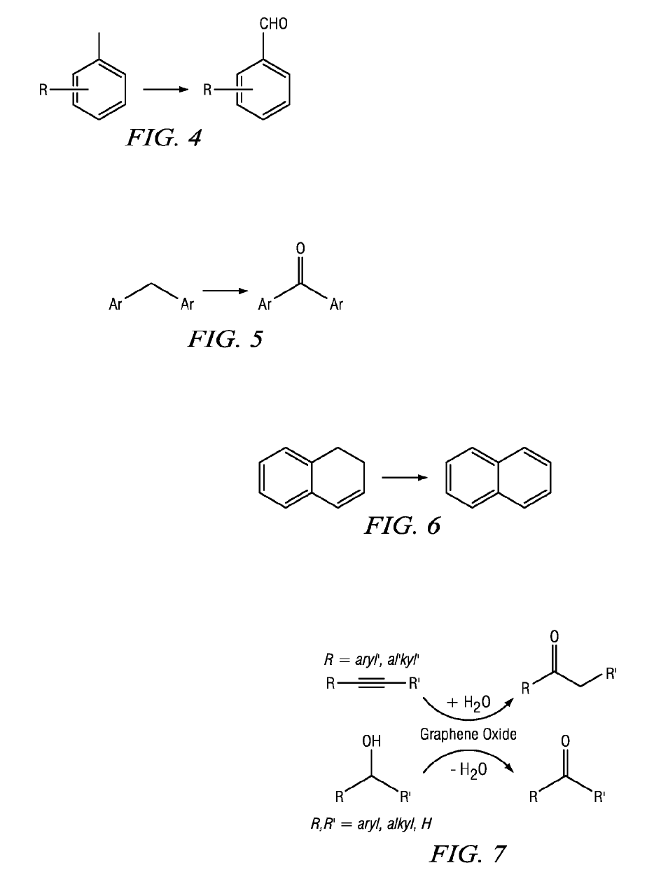

As shown in FIG. 4, graphene oxide or graphite oxide is used to catalyze the oxidation of methyl groups present on aryl rings, (e.g., methyl benzenes), to aldehydes. By way of example, many methyl benzenes, such as the model compound shown in FIG. 4, toluene, are very inexpensive. Oxidation of this substrate to benzyl alcohol, benzaldehyde, or benzoic acid is often difficult to perform, and almost invariably requires the use of metals. Accordingly, the carbocatalysts described herein (e.g., graphene oxide or graphite oxide) provide an expeditious, low cost route to effecting oxidations of methyl benzenes (toluenes) to the corresponding benzyl alcohols, benzaldehydes, or benzoic acids.

In additional embodiments, methyl groups present on saturated, partially unsaturated and/or unsaturated chains such as fatty acids, or rings cycloalkyl, heterocycloalkyl, or heteroaryl rings are also oxidized to the corresponding aldehydes. Other heterocycle-containing substrates and/or other cycloalkyl-containing substrates and/or other alkanes are expected to show similar reactivity. Examples of such substrates include: methyl-substituted heteroaryl rings such as imidazole, pyrazole, pyrrole, pyrrolidine, pyridine, piperidine, oxazole, thiazole, triazole (all isomers), tetrazole, thiophene, furan, thiofuran, indole, indoline, and others; cycloalkyls such as methylcyclohexane, methylcycloheptane (oxidized to the corresponding aldehydes), and others; heterocycloalkyls such as 2-methylpyrrolidine, 4-methylpiperidine, and the like; methyl groups in branched alkyls, and the like.

In a further aspect, for any of such methyl group containing substrates, perturbation of the electronic properties of the methyl group containing substrate (e.g., through the incorporation of electron-donating or -withdrawing substituents does not significantly affect the isolated yield of the desired product. Thus substrates comprising either electron donating groups (e.g., methoxy, dialkylamino, or any other electron donating group) or electron withdrawing groups (e.g., nitro, halo, or any other electron withdrawing group) are amenable to oxidation in the presence of carbocatalysts described herein. Thus the methods of oxidation in the presence of catalytically active carbocatalysts presented herein have wide synthetic utility.

In exemplary embodiments, reaction is carried out at a temperature of between 100° C. and 120° C. for a duration of 6-48 hours in the presence of between less than 1 wt % to 1000 wt % graphene oxide or graphite oxide.

It will be recognized that any combination of catalyst loadings, reaction times, solvent and reaction temperatures, as described in the sections above, are contemplated as within the scope of any of the embodiments described above or below.

### Oxidation of Disubstituted Methylenes to Ketones

As shown in FIG. 5, graphene oxide or graphite oxide is used to catalyze the oxidation of disubstituted methylenes, such as diarylmethanes, to ketones. Example disubstituted methylenes include diphenylmethane, which is oxidized to the corresponding diphenyl ketone.

In additional embodiments, methylene groups in saturated, partially unsaturated and/or unsaturated alkyl chains such as e.g., fatty acids, or rings such as cycloalkyl, heterocycloalkyl, or heteroaryl rings are also oxidized to the corresponding ketones. By way of example, heterocycle-containing molecules, such as 4-(p-nitrobenzyl)-pyridine (oxidized to the corresponding ketone) are oxidized in the presence of carbocatalysts described herein.

Other heterocycle-containing substrates and/or other cyclic substrates are expected to show similar reactivity. Examples of such substrates include: disubstituted methylenes where the methylene is substituted with: heteroaryl rings such as imidazole, pyrazole, pyrrole, pyrrolidine, pyridine, piperidine, oxazole, thiazole, triazole (all isomers), tetrazole, thiophene, furan, thiofuran, indole, indoline, and others; disubstituted methylenes where the methylene is substituted with cyclohexane, cycloheptane, cyclopropanes, cyclobutane and others (oxidized to the corresponding ketones), and others; disubstituted methylenes where the methylene is substituted with heterocycloalkyls such as 2-methylpyrrolidine, 4-methylpiperidine and the like.

In a further aspect, for any disubstituted methylenes, perturbation of the electronic properties of substrate disubstituted methylenes (e.g., through the incorporation of electron-donating or -withdrawing substituents does not significantly affect the isolated yield of the desired product. Thus substrates comprising either electron donating groups (e.g., methoxy, dialkylamino, or any other electron donating group) or electron withdrawing groups (e.g., nitro, halo, or any other electron withdrawing group) are amenable to oxidation in the presence of carbocatalysts described herein. Thus the methods of oxidation in the presence of catalytically active carbocatalysts presented herein have wide synthetic utility.

It will be recognized that any combination of catalyst loadings, reaction times, solvent and reaction temperatures, as described in the sections above, are contemplated as within the scope of any of the embodiments described above or below.

### Oxidation of Hydrocarbons

In some embodiments, a catalytically active carbocatalyst described herein functions as an oxidant for a hydrocarbon (e.g., methane, ethane, ethene or other hydrocarbons). In such situations, a hydrocarbon is oxidized to an alcohol when exposed to a catalytically active carbocatalyst for a suitable length of time and/or at a suitable temperature. In a further option, an alcohol oxidation product is further oxidized to an aldehyde or a ketone upon exposure to a catalytically activated graphene or graphite oxide for a longer duration of time and/or at a higher temperature. For example, methane is oxidized to methanol, or is optionally further oxidized to formaldehyde, or even further oxidized to formic acid.

It will be recognized that any combination of catalyst loadings, reaction times, solvent and reaction temperatures, as described in the sections above, are contemplated as within the scope of any of the embodiments described above or below.

### Oxidation of Alcohols to Ketones or Aldehydes

In alcohol oxidation, the graphene oxide or graphite oxide may function as a catalytic oxidant for the transformation of primary or secondary alcohols to their respective ketones or aldehydes. An example of one such reaction is shown in FIG. 7. The catalysis reaction is carried out by mixing graphene oxide or graphite oxide with the alcohol under conditions suitable to allow oxidation of the alcohol. The ketones or aldehydes formed is removed during the reaction or afterwards. Ketones alone is formed, or aldehydes alone is formed, or combinations of ketones and aldehydes is formed. Oxygen, such as ambient oxygen, or another molecule may serve as the terminal oxidant in the alcohol oxidation reaction. The oxidation reaction may proceed using radical species.

According to specific embodiments, the alcohol is aromatic or aliphatic. The conversion rate is higher for aromatic alcohols than for their aliphatic analogs. According to another embodiment, the alcohol is a benzylic alcohol.

In additional embodiments, alcoholic groups present on saturated, partially unsaturated and/or unsaturated alkyl chains, or rings such as cycloalkyl, heterocycloalkyl, or heteroaryl rings are also oxidized to the corresponding aldehydes. By way of example, heterocycle-containing molecules, such as 3-indolyl methanol, 2-thiophenemethanol are oxidized in the presence of carbocatalysts described herein to the corresponding aldehydes.

Other heterocycle-containing substrates and/or other cyclic substrates are expected to show similar reactivity. Examples of such substrates include: methanol-substituted heteroaryl rings such as imidazole, pyrazole, pyrrole, pyrrolidine, pyridine, piperidine, oxazole, thiazole, triazole (all isomers), tetrazole, thiophene, furan, thiofuran, indole, indoline, and others; methanol-substituted cycloalkyls such as cyclopropane, cyclobutane, cyclopropane, cyclohexane, cycloheptane (oxidized to the corresponding aldehydes), and others; methanol-substituted heterocycloalkyls such as pyrrolidine, 4-piperidine and the like.

In a further aspect, for methanol-substituted compounds, perturbation of the electronic properties of methanol-substitued substrates (e.g., through the incorporation of electron-donating or -withdrawing substituents does not significantly affect the isolated yield of the desired product. Thus substrates comprising either electron donating groups (e.g., methoxy, dialkylamino, or any other electron donating group) or electron withdrawing groups (e.g., nitro, halo, or any other electron withdrawing group) are amenable to oxidation in the presence of carbocatalysts described herein. Thus the methods of oxidation in the presence of catalytically active carbocatalysts presented herein have wide synthetic utility.

In certain exemplary embodiments, the oxidation reaction is carried out at a temperature above 75° C., more particularly between 75° C. and 150° C., or at a temperature above 100° C., more particularly between 100° C. and 150° C. The amount of graphene oxide or graphite oxide used is at least 50 wt %, more particularly between 50 wt % and 200 wt %. The conversion of alcohol to a ketone or aldehyde is at least 85%, more particularly at least 99%. The ratio of ketone or aldehyde:acid in the reaction product is 92:7 or better.

In another exemplary embodiment, the reaction is carried out with 200 wt % catalyst at 100° C. for 24 hours.

It will be recognized that any combination of catalyst loadings, reaction times, solvent and reaction temperatures, as described in the sections above, are contemplated as within the scope of any of the embodiments described above or below.

### Oxidation of Thiols and Sulfides

Carbocatalysts such as graphene oxide or graphite oxide facilitate the oxidation of thiols and sulfides to their corresponding disulfides and sulfoxides, respectively. Examples of such reactions are shown in FIG. 10. According to specific embodiments, the thiol or sulfide is aromatic or aliphatic.

In additional embodiments, thiol or sulfide groups present on saturated, partially unsaturated and/or unsaturated alkyl chains, or rings such as cycloalkyl, heterocycloalkyl, or heteroaryl rings are also oxidized to the corresponding disulfides or sulfoxides. By way of example, thiophenol is oxidized to the corresponding disulfide, diphenylsulfide is oxidized to the corresponding sulfoxide in the presence of graphene oxide or graphite oxide.

Other heterocycle-containing substrates and/or other cyclic substrates are expected to show similar reactivity. Examples of such substrates include: thiol-substituted heteroaryl rings such as imidazole, pyrazole, pyrrole, pyrrolidine, pyridine, piperidine, oxazole, thiazole, triazole (all isomers), tetrazole, thiophene, furan, thiofuran, indole, indoline, and others; methanol-substituted cycloalkyls such as cyclopropane, cyclobutane, cyclopropane, cyclohexane, cycloheptane (oxidized to the corresponding aldehydes), and others; methanol-substituted heterocycloalkyls such as pyrrolidine, 4-piperidine and the like.

In a further aspect, for thiol-substituted compounds, perturbation of the electronic properties of substrate thiols (e.g., through the incorporation of electron-donating or -withdrawing substituents does not significantly affect the isolated yield of the desired product. Thus substrates comprising either electron donating groups (e.g., methoxy, dialkylamino, or any other electron donating group) or electron withdrawing groups (e.g., nitro, halo, or any other electron withdrawing group) are amenable to oxidation in the presence of carbocatalysts described herein. For example, both electron rich (entries 4-7) and electron deficient thiols (entries 8-10) gave excellent isolated yields of the respective disulphides (94-99%). No over-oxidation (including N-oxidation, in the case of 2-aminothiophenol) was observed in any of the reactions performed, as has been observed with chromic potassium sulphate (CrK(SO₄)₂.12H₂O) and other transition metal-based oxidation catalysts. Thus the methods of oxidation in the presence of catalytically active carbocatalysts presented herein have wide synthetic utility.

It will be recognized that any combination of catalyst loadings, reaction times, solvent and reaction temperatures, as described in the sections above, are contemplated as within the scope of any of the embodiments described above or below.

### Oxidation of Nitrogen Containing Compounds

In one embodiment, the carbocatalysts described herein, e.g., graphene oxide or graphite oxide, are suitable for oxidation of nitrogen containing compounds such as amines (e.g., triethylamine, triphenylamine or other stable tertiary amines) or N-heterocycles (e.g., pyridine, piperidine, pyrrolidine, etc.) to corresponding N-oxides. Oxidation to other nitrogen functional groups is anticipated as well, including oxidations of amines to nitro, nitroso, diazo, and diazonium species. In one embodiment, oxidation of primary amines (e.g., benzyl amine) yields corresponding aldehydes (e.g., benzaldehyde from benzyl amine).

In a further aspect, for nitrogen containing compounds, perturbation of the electronic properties of substrates (e.g., through the incorporation of electron-donating or -withdrawing substituents does not significantly affect the isolated yield of the desired product. Thus substrates comprising either electron donating groups (e.g., methoxy, dialkylamino, or any other electron donating group) or electron withdrawing groups (e.g., nitro, halo, or any other electron withdrawing group) are amenable to oxidation in the presence of catalytically active carbocatalysts described herein.

It will be recognized that any combination of catalyst loadings, reaction times, solvent and reaction temperatures, as described in the sections above, are contemplated as within the scope of any of the embodiments described above or below.

### Oxidation of Imines

In additional embodiments, reaction of imines with graphene oxide or graphite oxide is expected to afford other oxidation products, such as aldehydes or carboxylic acids. In certain cases, isolation of the imine N-oxide oxidation product is possible (e.g., upon removal of water prior to hydrolysis or during preparation of a particularly stable imine).

It will be recognized that any combination of catalyst loadings, reaction times, solvent and reaction temperatures, as described in the sections above, are contemplated as within the scope of any of the embodiments described above or below.

### Dehydrogenation Reactions

As shown in FIG. 6, graphene oxide or graphite oxide is used to catalyze a dehydrogenation reactions, such as the aromatization of cyclic, hydrogenated species, for example the aromatization of tetralin to naphthalene or dihydronapthalene to napthalene. This dehydrogenation chemistry is useful when combining this reaction with other reactions.

Other heterocycle-containing substrates and/or other cyclic substrates are expected to show similar reactivity. Examples of such substrates include: tetrahydroquinolines, 1,2-dihydroquinazolines, and the like.

In a further aspect, perturbation of the electronic properties of substrates (e.g., through the incorporation of electron-donating or -withdrawing substituents does not significantly affect the isolated yield of the desired product. Thus substrates comprising either electron donating groups (e.g., methoxy, dialkylamino, or any other electron donating group) or electron withdrawing groups (e.g., nitro, halo, or any other electron withdrawing group) are amenable to dehydrogenation in the presence of carbocatalysts described herein.

Thus the methods of dehydrogenation in the presence of catalytically active carbocatalysts presented herein have wide synthetic utility.

In exemplary embodiments, dehydrogenation reaction is carried out at a temperature of between 100° C. and 120° C. for a duration of 6-48 hours in the presence of less than 1 wt % to 1000 wt % graphene oxide or graphite oxide.

It will be recognized that any combination of catalyst loadings, reaction times, solvent and reaction temperatures, as described in the sections above, are contemplated as within the scope of any of the embodiments described above or below.

### Hydration Reactions

### Hydration of Alkynes to Ketones

In alkyne hydration, the graphene oxide or graphite oxide may function as a catalytic oxidant for the transformation of unsubstituted, mono- or disubstituted alkynes to the corresponding ketones. The catalysis reaction is carried out by mixing graphene oxide or graphite oxide with the alkyne under conditions suitable to allow hydration of the alkyne. The ketones formed is removed during the reaction or afterwards. An example alkyne hydration is show in FIG. 8.

In specific embodiments, the alkyne is aromatic or aliphatic. The conversion rate is higher for aromatic alkynes than for their aliphatic analogs. The conversion rate for alkynes is at least 52%, more particularly at least 98%.

In an exemplary embodiment, the reaction is carried out with 200 wt % catalyst at 100° C. for 24 hours.

It will be recognized that any combination of catalyst loadings, reaction times, solvent and reaction temperatures, as described in the sections above, are contemplated as within the scope of any of the embodiments described above or below.

### Hydration of Alkenes to Alcohols

In alkne hydration, the graphene oxide or graphite oxide may function as a catalytic oxidant for the transformation of alkenes to the corresponding alcohols. The catalysis reaction is carried out by mixing graphene oxide or graphite oxide with the alkyne under conditions suitable to allow hydration of the alkyne. The ketones formed is removed during the reaction or afterwards.

In specific embodiments, the alkene is unsubstituted, mono-, di- tri- or tetra-substituted alkene and is aromatic or aliphatic. In some embodiments, the alkene is a cis alkene. In other embodiments, the alkene is a trans alkene. The conversion rate is higher for aromatic alkynes than for their aliphatic analogs.

In an exemplary embodiment, the reaction is carried out with 200 wt % catalyst at 100° C. for 24 hours.

It will be recognized that any combination of catalyst loadings, reaction times, solvent and reaction temperatures, as described in the sections above, are contemplated as within the scope of any of the embodiments described above or below.

### Hydration of Nitriles

In nitrile hydration, the graphene oxide or graphite oxide functions as a catalytic oxidant for the transformation of nitriles to the corresponding carboxylates and/or carboxylic acids. The catalysis reaction is carried out by mixing graphene oxide or graphite oxide with the nitrile under conditions suitable to allow hydration of the nitrile. The carboxylic acid formed is removed during the reaction or afterwards.

It will be recognized that any combination of catalyst loadings, reaction times, solvent and reaction temperatures, as described in the sections above, are contemplated as within the scope of any of the embodiments described above or below.

### Hydration of Imines

In imine hydration, the graphene oxide or graphite oxide functions as a catalytic oxidant for the transformation of imines to the corresponding amines. The catalysis reaction is carried out by mixing graphene oxide or graphite oxide with the imine under conditions suitable to allow hydration of the imine. The amine formed is removed during the reaction or afterwards.

It will be recognized that any combination of catalyst loadings, reaction times, solvent and reaction temperatures, as described in the sections above, are contemplated as within the scope of any of the embodiments described above or below.

### Condensation Reactions and Auto-Tandem Oxidation-Hydration Aldol Coupling

Graphene oxide or graphite oxide is used to catalyze the condensation reaction of ketones and aldehydes to chalcones. According to another embodiment, shown in FIG. 9, an auto-tandem oxidation-hydration-aldol coupling reaction is used. In particular, graphene oxide or graphite oxide is used to catalyze the formation of chalcone via an auto-tandem reaction in one vessel from alkynes and alcohols. The alkynes and alcohols are hydrated or oxidized to their corresponding methyl ketones or aldehydes respectively, with the reaction being catalyzed by the graphene oxide or graphite oxide. Then the ketones or aldehydes undergo a Claisen-Schmidt condensation, also catalyzed by the graphene oxide or graphite oxide, to form a range of chalcone products. Thus, in the overall chalcone formation, the graphene oxide or graphite oxide catalyzes alcohol oxidation, alkyne hydration, and C—C bond-forming condensation.

The direct coupling of alcohols and alkynes in auto-tandem synthesis of chalcones is most efficient in many situations, but each of these reactions taking place in the auto-tandem synthesis may also be performed separately. For example, methods of alcohol oxidation and alkyne hydration are described above. The products of those reactions may then be reacted in the presence of graphene oxide or graphite oxide to form a chalcone. Ketones and aldehydes from other may also be combined in the presence of graphene oxide or graphite oxide to form a chalcone.

Example alcohols include, benzyl alcohol, p-methoxybenzyl alcohol, and p-nitrobenzyl alcohol. Example alkynes include phenylacetylene, p-methoxyphenylacetylene, and p-nitrophenylacetylene were explored. Example ketones include acetophenone, p-methoxyacetophenone, and p-nitroacetophene were explored. Example aldehydes include benzaldehyde, p-methyoxybenzaldehyde, and p-nitrobenzaldehyde in these reactions. For preparing chalcones, other substituted benzaldehydes might be employed. Likewise, other alcohols, alcohols, or methyl ketones might be employed.

In an exemplary embodiment, a condensation reaction is carried out at a temperature of between 80-150° C. for a duration of 14-24 hours in the presence of less than 1 wt % to 1000 wt % graphene oxide or graphite oxide.

Reaction conditions are optimized for any reaction described herein to increase yield. They may also be optimized to reduce competing side reactions. In certain embodiments, the yield is at least 50%, at least 60%, at least 80%, at least 90%, or at least 95%. The turnover number for the reaction is on the order of 10⁻² (expressed in moles of product per mass of catalyst).

In exemplary embodiments, the reactions is carried out at a temperature 25° C., or above 75° C., more particularly between 75° C. and 150° C., or at a temperature above 100° C., more particularly between 100° C. and 150° C. In certain embodiments, the reaction temperature is between 100° C. and 120° C.

In some embodiments, reaction time is between 3-144 hours. The time required may vary with the type of reaction catalyzed.

It will be recognized that any combination of catalyst loadings, reaction times, solvent and reaction temperatures, as described in the sections above, are contemplated as within the scope of any of the embodiments described above or below.

Although specific reaction conditions, turnovers and yields are specified herein, the above conditions, turnover and yields may apply to any reaction catalyzed by graphene oxide or graphite oxide.

### EXAMPLES

The present invention may be better understood through reference to the following examples. These examples are included to describe exemplary embodiments only and should not be interpreted to encompass the entire breadth of the invention.

### Example 1

### Preparation of Graphene Oxide or Graphite Oxide Catalyst

The graphene oxide or graphite oxide used in some experiments contained in these examples was prepared according to the following method. Others were prepared using the Staudenmaier method. Both methods resulted in a suitable catalyst.

A modified Hummers method was used to prepare the graphite oxide. A 100 mL reaction flask was charged with natural flake graphite (3.0 g; SP-1, Bay Carbon Inc. or Alfa Aesar [99%; 7-10 μm]), concentrated sulfuric acid (75 mL), and a stir bar, and then cooled on an ice bath. The flask was then slowly charged with KMnaₜ (9.0 g) over 2 h which afforded a dark colored mixture. The rate of addition was controlled carefully to prevent the temperature of the suspension from exceeding 20° C. After stirring at 0° C. for 1 h, the mixture was heated at 35° C. for 0.5 h. The flask was then cooled to room temperature and the reaction was quenched by pouring the mixture into 150 mL of ice water and stirred for 0.5 h at room temperature. The mixture was further diluted to 400 mL with water and treated with a 30% aqueous solution of hydrogen peroxide (7.5 mL). The resulting vibrant yellow mixture was then filtered and washed with an aqueous HCl solution (6.0 N) (800 mL) and water (4.0 L). The filtrate was monitored until the pH value was neutral and no precipitate was observed upon the addition of aqueous barium chloride or silver nitrate to the filtrate. The filtered solids were collected and dried under high vacuum to afford the desired product (5.1 g) as a dark brown powder. Spectral data matched literature values.

### Example 2

### Preparation of Graphite Oxide

A 100 mL reaction flask is charged with natural flake graphite (6.0 g; SP-1, Bay Carbon Inc. or Alfa Aesar [99%; 7-10 μm]), concentrated sulfuric acid (25 mL), K₂S₂O₈ (5 g), P₂O₅ (5 g), and a stir bar, and then the mixture is heated at 80° C. for 4.5 h. The mixture is then cooled to room temperature. Next, the mixture is diluted with water (1 L) and left undisturbed for a period of about 8-10 hours. The pretreated graphite is collected by filtration and washed with water (0.5 L). The precipitate is dried in air for 1 day and transferred to concentrated H₂SO₄ (230 mL). The mixture is then slowly charged with KMnO₄ (30 g) over 2 h, which affords a dark colored mixture. The rate of addition is carefully controlled to prevent the temperature of the suspension from exceeding 10° C. The mixture is stirred at 0° C. for 1 h. The mixture is then heated at 35° C. for 2 h. The flask is then cooled to room temperature and the reaction is quenched by pouring the mixture into 460 mL of ice water and stirred for 2 h at room temperature. The mixture is further diluted to 1.4 L with water and treated with a 30% aqueous solution of hydrogen peroxide (25 mL). The resulting vibrant yellow mixture is then filtered and washed with an aqueous HCl solution (10%) (2.5 L) and then with water. The filtrate is monitored until the pH value is neutral and no precipitate is observed upon the addition of aqueous barium chloride or silver nitrate to the filtrate. The filtered solids are collected and dried under high vacuum to provide a product (11 g) as a dark brown powder.

### Example 3

### Preparation of Graphite Oxide

A 250 mL reaction flask is charged with natural flake graphite (1.56 g; SP-1 Bay Carbon Inc. or Alfa Aesar [99%; 7-10 μm]), 50 mL of concentrated sulfuric acid, 25 mL fuming nitric acid, and a stir bar, and then cooled in an ice bath. The flask is then charged with NaClO₃ (3.25 g; note: in some cases NaClO₃ is preferable over KClO₃ due to the aqueous insolubility of KClO₄ that may form during the reaction) under stirring. Additional charges of NaClO₃ (3.25 g) are performed every hour for 11 consecutive hours per day. This procedure is repeated for 3 d. The resulting mixture is poured into 2 L deionized water. The heterogeneous dispersion is then filtered through a coarse fritted funnel or a nylon membrane filter (0.2 μm, Whatman) and the isolated material is washed with additional deionized water (3 L) and 6 N HCl (1 L). The filtered solids are collected and dried under high vacuum to provide a product (3.61 g) as a dark brown powder.

### Example 4

### Preparation of Graphene Oxide

A graphene substrate is provided in a reaction chamber. The substrate does not exhibit one or more FT-IR peaks at 3150 cm⁻¹, 1685 cm⁻¹, 1280 cm⁻¹ or 1140 cm⁻¹. Next, plasma excited species of oxygen are directed from a plasma generator into the reaction chamber and brought in contact with an exposed surface of the graphene substrate. The graphene substrate is exposed to the plasma excited species of oxygen until an FT-IR spectrum of the substrate shows one or more peaks at 3150 cm⁻¹, 1685 cm⁻¹, 1280 cm⁻¹ or 1140 cm⁻¹. The graphene substrate has a layer of graphene oxide on the exposed surface of the graphene substrate.

### Example 5

### Oxidation of Alkenes Using Graphene Oxide or Graphite Oxide

Graphene oxide or graphite oxides may also be used to oxidize alkenes. Alkenes are also sometimes referred to in the art as olefins. The general reaction scheme for oxidation of cis-stilbene (an alkene) to benzil (a dione) may be seen in FIG. 2. In one test, after reaction of cis-stilbene with 20 wt % graphene oxide or graphite oxide for 24 hours at 100° C., a 56% conversion was obtained, which, after isolation of the benzil, resulted in a 49% yield. No benzoin or other oxidation products were observed in the reaction mixture.

In a typical preparation, a 7.5 mL vial was charged with graphene oxide or graphite oxide (200 mg), cis-stilbene (50 mg), CHCl₃ (0.5 mL) and a magnetic stir bar. The vial was then sealed with a Teflon-lined cap under ambient atmosphere and heated at 100° C. for 24 h. After the reaction was complete, the mixture was cooled to room temperature and washed with CH₂Cl₂ (50 mL). The filtrate was collected and the solvent was evaporated to obtain the crude product, which was then purified by silica chromatography (CH₂Cl₂ or CH₂Cl₂/hexanes as the eluent) and the solvent was removed under reduced pressure to obtain the desired product. All isolated products matched spectroscopic data reported previously in the literature. This reaction is shown in FIG. 3.

As summarized in Tables 1 and 2, the oxidation of cis-stilbene to benzil was optimized with respect to the loading of graphene oxide or graphite oxide and the reaction temperature. In general, increasing the loading of graphene oxide or graphite oxide used in the reaction was found to increase the yield of the benzil product, though no improvement was observed beyond 400 wt %. At low temperatures (<60° C.), minimal conversion was observed, regardless of graphene oxide or graphite oxide loading; however, at temperatures at or above 100° C., yields also decreased, presumably due to thermal degradation of the graphene oxide or graphite oxide substrate. The use of a nitrogen atmosphere, in lieu of air, was also explored, but minimal effects on the product yield were observed.

TABLE 1
Optimization of the oxidation of cis-stilbene to benzil:
effect of graphene oxide or graphite oxide loading.^{a}

| Entry | GO | Atmosphere | Isolated yield (%) |
|---|---|---|---|
| 1 | 100 mg | ambient | 49 |
| 2 | 150 mg | ambient | 57 |
| 3 | 200 mg | ambient | 68 |
| 4 | 100 mg | N₂ | 44^{b} |
| 5 | 200 mg | N₂ | 58^{b} |

^{a}All reactions were performed using 50 mg of cis-stilbene and 0.5 mL of CHCl₃ heated at 100° C. for 24 h.
^{b}Yield determined by ¹H NMR spectroscopy.

TABLE 2
Optimization of the oxidation of cis-stilbene to benzil: effect
of graphene oxide or graphite oxide loading and temperature.^{a}

| Entry | GO | Temperature | Isolated yield (%) |
|---|---|---|---|
| 1 | 25 mg | 60° C. | 5 |
| 2 | 25 mg | 80° C. | 8 |
| 3 | 25 mg | 100° C. | 11 |
| 4 | 100 mg | 100° C. | 49 |

^{a}All reactions were performed using 50 mg of cis-stilbene (neat) and heated at 100° C. for 24 h.

Ultimately, optimized conditions were determined (50 mg cis-stilbene, 200 mg graphene oxide or graphite oxide, 0.5 mL CHCl₃, 100° C., 24 h) and benzil was obtained in 68% isolated yield. Using these conditions, the reactivity of graphene oxide or graphite oxide toward other substrates was explored, beginning with a series of functionalized cis-stilbenes.

As summarized in Table 3, the use of substrates featuring strongly electron-donating or moderately electron-withdrawing groups did not improve the isolated yield of the respective dione product (entries 1-3; 12-50%). Additionally, cis-stilbenes that possessed strongly electron-withdrawing groups, such as cis-dinitrostilbene (entry 4), afforded the desired products in only modest yields (25%). However, weakly electron-donating cis-stilbenes (entry 5) gave products in an improved isolated yield (68%). Nevertheless, these results are substantial improvement on existing techniques as the oxidation of stilbenes or similar 1,2-disubstituted olefins to their corresponding diones typically requires the use of metal catalysts, such as selenium(IV) oxide or chromate species.

TABLE 3
Oxidation of various substituted cis-stilbenes using graphene oxide or
graphite oxide.^{a}

| Entry | Starting material | Product | Isolated yield |
|---|---|---|---|
| 1 | 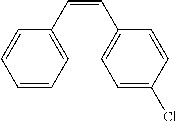 [SMILES: Clc1ccc(/C=C\c2ccccc2)cc1] | 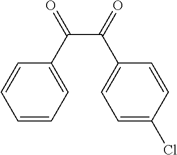 [SMILES: O=C(C(=O)c1ccc(Cl)cc1)c1ccccc1] | 50% |
| 2 | 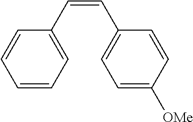 [SMILES: COc1ccc(/C=C\c2ccccc2)cc1] | 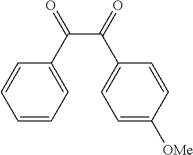 [SMILES: COc1ccc(C(=O)C(=O)c2ccccc2)cc1] | 38% |
| 3 | 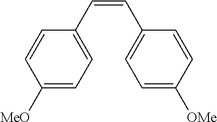 [SMILES: COc1ccc(/C=C\c2ccc(OC)cc2)cc1] | 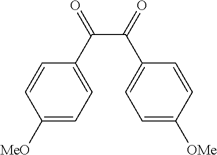 [SMILES: COc1ccc(C(=O)C(=O)c2ccc(OC)cc2)cc1] | 48% |
| 4 | 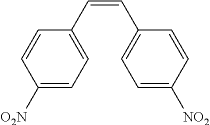 [SMILES: O=[N+]([O-])c1ccc(/C=C\c2ccc([N+](=O)[O-])cc2)cc1] | 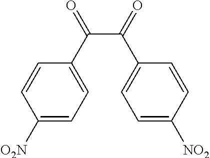 [SMILES: O=C(C(=O)c1ccc([N+](=O)[O-])cc1)c1ccc([N+](=O)[O-])cc1] | 25% |
| 5 | 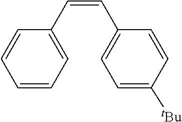 [SMILES: CC(C)(C)c1ccc(/C=C\c2ccccc2)cc1] | 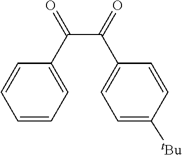 [SMILES: CC(C)(C)c1ccc(C(=O)C(=O)c2ccccc2)cc1] | 68% |
| 6 | 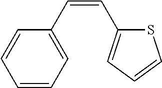 [SMILES: C(=C/c1cccs1)/c1ccccc1] | 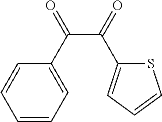 [SMILES: O=C(C(=O)c1cccs1)c1ccccc1] | 12% |

^{a}All reactions were performed in 0.5 mL CHCl₃ using 50 mg of the substrate and 200 mg (400 wt %) of graphene oxide or graphite oxide at 100° C. for 24 h in Teflon-sealed vials.

### Example 6

### Oxidation of Hydrocarbons Possessing Methyl or Methylene Groups

In a typical preparation, a 7.5 mL vial was charged with graphene oxide or graphite oxide (100 mg), substrate (50 mg), CHCl₃ (0.5 mL) and a magnetic stir bar. The vial was then sealed with a Teflon-lined cap under ambient atmosphere and heated at 70° C., 120° C. or 120° C. for 24 h. The reaction mixture was then cooled to room temperature and washed with CH₂Cl₂ (50 mL). The filtrate was collected and the solvent was removed under vacuum to obtain the crude product, which was then purified by silica chromatography (CH₂Cl₂, hexanes, EtOAc/CH₂Cl₂ or CH₂Cl₂/hexanes as the eluent). The oxidation reactions involving toluene, 4-nitrotoluene, cyclohexadiene, cyclohexene and cyclohexane were performed using CDCl₃ instead of CHCl₃ and conversions were determined directly by ¹H NMR spectroscopy. All products matched spectroscopic data reported previously in the literature.

As summarized in Table 4, various substrates with benzylic methylene groups were successfully converted to their corresponding ketone and unsaturated products using graphene oxide or graphite oxide (conditions: 50 mg substrate, 100 mg graphene oxide or graphite oxide, 0.5 mL CHCl₃, 120° C., 24-48 h). For some substrates, such as diphenylmethane (entry 1), oxidation resulted in the formation of the respective ketone. In other cases, such as that of dihydrophenanthrene (entry 6), the loss of hydrogen and subsequent aromatization was observed. In general, the oxidation of substrates with doubly activated benzylic positions afforded the corresponding ketone products in high yield (e.g., 72% in the case of diphenylmethane). Conversely, substrates such as bibenzyl (entry 2), wherein each methylene is only activated by a single phenyl substituent, afforded decreased yields of product (17%). The reduced yield of benzil from bibenzyl may be due to the substrate's relatively high conformational freedom, although diphenylmethane (entry 1) was oxidized to its respective carbonyl in higher yield than fluorene (entry 3). Dihydrophenanthrene, which may be considered a rigid analogue of bibenzyl, also underwent dehydrogenation. Collectively, the results described above suggest that benzylic activation may be the primary factor in determining a substrate's susceptibility to C—H activation when graphene oxide or graphite oxide is used as the oxidant.

¹H NMR spectroscopic analysis of the crude reaction mixtures showed no by-products for nearly all the substrates explored in this study. The only exception was 9,10-dihydroanthracene (entry 5), which revealed a mixture of anthracene and anthraquinone (isolated in 49% and 31% yield, respectively). In comparison, prior experiments by others showed the oxidative dehydrogenation of 9,10-dihydroanthracene to anthracene when multi-walled carbon nanotubes (MWCNTs), a carbon-based material related to graphene oxide or graphite oxide, were used as the oxidation catalyst. (Bégin, D.; Ulrich, G.; Amadou, J.; Su, D. S.; Pham-Huu, C.; Ziessel, R. J. Mol. Catal. A: Chem. 2009, 302, 119-123; Fu, P. P.; Harvey, R. G. Chem. Rev. 1978, 78, 317-361.) Virtually no anthraquinone was detected in the product mixture obtained from the MWCNT-catalyzed reaction, a result which suggests that graphene oxide or graphite oxide's reactivity extends beyond that of MWCNTs. For example, using graphene oxide or graphite oxide, two substrates related to 9,10-dihydroanthracene, tetralin (entry 7) and 1,2-dihydronaphthalene (entry 8), were subjected to identical reaction conditions as those described above (50 mg substrate, 100 mg GO,¹⁷ 0.5 mL CHCl₃, 120° C., 24-48 h). Naphthalene was isolated as the sole product in 24% and 26% yield, respectively, in both reactions; the corresponding dione (naphthaquinone) was not observed.

In addition to the conversion of diaryl methylenes to their respective ketones, graphene oxide or graphite oxide was also found to oxidize substituted methyl benzenes to their corresponding aldehydes (entries 9-11), albeit in relatively low yields. In general, the incorporation of strongly electron-donating groups, such as 4-methylanisole (entry 9), afforded the corresponding aldehyde product in higher isolated yields compared to methyl benzenes bearing neutral or electron-withdrawing groups.

TABLE 4
Oxidation of various hydrocarbons using graphene oxide or graphite
oxide.^{a}

| | | | Isolated |
|---|---|---|---|
| Entry | Starting Material | Product | Yield % |
| 1 | 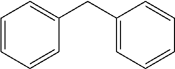 [SMILES: c1ccc(Cc2ccccc2)cc1] | 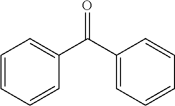 [SMILES: O=C(c1ccccc1)c1ccccc1] | 72% |
| 2 | 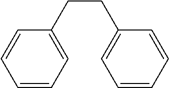 [SMILES: c1ccc(CCc2ccccc2)cc1] | 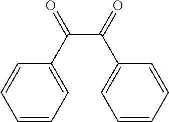 [SMILES: O=C(C(=O)c1ccccc1)c1ccccc1] | 17% |
| 3 | 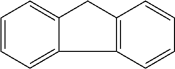 [SMILES: c1ccc2c(c1)Cc1ccccc1-2] | 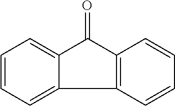 [SMILES: O=C1c2ccccc2-c2ccccc21] | 59% |
| 4 | 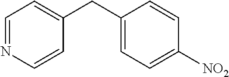 [SMILES: O=[N+]([O-])c1ccc(Cc2ccncc2)cc1] | 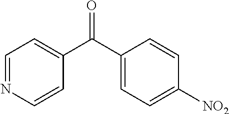 [SMILES: O=C(c1ccncc1)c1ccc([N+](=O)[O-])cc1] | 80% |
| 5 | 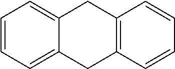 [SMILES: c1ccc2c(c1)Cc1ccccc1C2] | 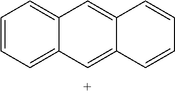 [SMILES: c1ccc2cc3ccccc3cc2c1] | 49% |
| | | 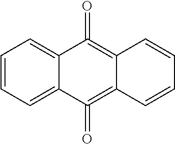 | 31% |
| 6 | 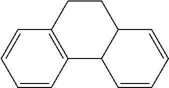 [SMILES: C1=CC2CCc3ccccc3C2C=C1] | 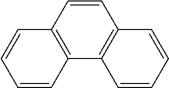 [SMILES: c1ccc2c(c1)ccc1ccccc12] | 85% |
| 7 | 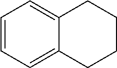 | 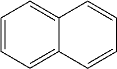 | 24% |
| 8 | 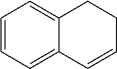 | 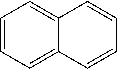 | 26% |
| 9 | 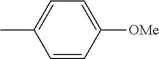 | 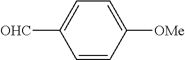 | 16% |
| 10 | 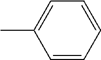 | 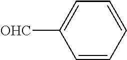 | 6%^{[b]} |
| 11 | 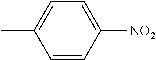 | 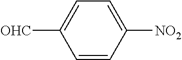 [SMILES: O=Cc1ccc([N+](=O)[O-])cc1] | 4%^{[b]} |

^{a}All reactions were performed in 0.5 mL CHCl₃ using 50 mg of the substrate and 100 mg (200 wt %) of graphene oxide or graphite oxide at 120° C. for 24 h in Teflon-sealed vials.
^{[b]}Yield determined by ¹H NMR spectroscopy.

### Example 7

### Oxidation of Alcohols

Various alcohols may be oxidized to ketones or aldehydes using a graphene or graphit oxide catalyst. Specific examples are described below.

Neat benzyl alcohol (PhCH₂OH) was heated to 100° C. in the presence of 20 wt % graphene oxide or graphite oxide for 14 hours under ambient conditions. The general reaction scheme is shown in FIG. 7. As FIG. 8 indicates, acceptable variations in weight percentage graphene oxide or graphite oxide are between 5-200 wt %. Acceptable variations in reaction temperature are between 25-100° C. Acceptable variations in reaction time are between 3-144 hours.

Subsequent filtering of the reaction product afforded a mixture of benzylaldehyde (PhCHO):PhCH₂OH in a 1:3 molar ratio, as determined by ¹H NMR spectroscopy. No over-oxidation to benzoic acid (PhCO₂H) or other by-products was detected under these conditions. NMR analysis and titrimetry of the reaction product mixture showed that water was released during the reaction.

Atomic absorption and inductively coupled plasma mass spectrometry showed that the graphene oxide or graphite oxide catalyst used contained trace or no amounts of metals, indicating that the graphene oxide or graphite oxide and not a metal functioned as a catalyst. The catalytic activity of graphene oxide or graphite oxide was further confirmed by the inability of corresponding flake graphite to catalyze the alcohol oxidation reaction under identical conditions.

The graphene oxide or graphite oxide used in the oxidation reaction was separated from the reaction mixture and filtered. The filtered material was characterized using FT-IR (KBr) and showed an attenuated signal relative to other peaks in the spectrum at υ=3150 cm⁻¹, corresponding to the O—H stretch as well as no diagnostic signals at 1685 cm⁻¹, corresponding to the C═O stretch and 1280 cm⁻¹ or 1140 cm⁻¹, corresponding to epoxide absorbances. C═O and epoxide absorbance signals were seen in the unused catalyst. The FT-IR spectrum also showed signals at 1650 cm⁻¹ and 1500 cm⁻¹, corresponding to the presence of aromatic and olefin species. These signals were not seen in the unused catalyst. Power conductivity in the used graphene oxide or graphite oxide was also measured to be 15 S/m, which was a significant increase from the 2.2×10⁻⁵ S/m measured in the unused catalyst. Collectively, these results suggest that the graphene oxide or graphite oxide catalyst undergoes a partial reduction during the alcohol oxidation reaction. The used graphene oxide or graphite oxide is similar to r-graphene oxides or graphenes that have been synthesized by other methods.

The catalytic function of the graphene oxide or graphite oxide, as opposed to direct oxidation of the alcohol by the graphene oxide or graphite oxide, was also confirmed. Under ambient conditions, when the graphene oxide or graphite oxide reacts with the alcohol as a catalyst, ambient oxygen is the terminal oxidant. In particular, the PhCH₂OH to PhCHO reaction with a graphene oxide or graphite oxide was performed under a nitrogen atmosphere. After 24 hours of reaction time at 100° C., an aliquot removed from the reaction mixture contained less than 5 mol % PhCHO, indicating that the alcohol oxidation reaction was not able to proceed in the absence of oxygen. In contrast, if the graphene oxide or graphite oxide directly oxidized the alcohol, oxygen would not be needed and a higher PhCHO yield would be expected.

The reaction was allowed to continue for another 24 hours at 100° C. and another aliquot was tested, showing only 23 mol % conversion of the alcohol to PhCHO, further confirming the catalytic action of the graphene oxide or graphite oxide.

Furthermore, when the graphene oxide or graphite oxide was separated from the reaction mixture by filtration, it was found to retain its oxidative properties and could be reused for multiple reaction cycles.

The low yield under a nitrogen atmosphere also indicates that, in situations where it is desirable to diminish the oxidative capacity of the catalyst, an atmosphere lacking oxygen or other final oxidants may be used.

To determine the role of radical species in the oxidation process, neat PhCH₂OH was treated with 20 wt % graphene oxide or graphite oxide and 20 wt % butylated hydroxytolulene (BHT). BHT affects the formation of radical species by forming a more stable, non-reactive radical that arrests the oxidation reaction or by inhibiting or preventing the formation of radicals. After 24 hours reaction time at 100° C., less than 5 mol % conversion of the alcohol to PhCHO was observed. However, when a 1:1:1 mass ratio of graphene oxide or graphite oxide:PhCH₂OH:BHT was heated for 14 hours at 100° C., a 26 mol % conversion of the alcohol to PhCHO was observed. This indicates that BHT likely does not inhibit the graphene oxide or graphite oxide's ability to oxidize the alcohol, but rather impedes the oxidation of the reduced catalyst. This assessment was further confirmed by heating graphene oxide or graphite oxide in the presence of cyclopropylcarbinol, which reacts in a very specific manner, evidenced by the formation of olefinic species, in the present of radicals. These olefinic species may be readily identified spectroscopically. This resulted in the formation of number of products including olefinic species, but no aldehydes. Similar results are obtained in chromic-acid mediated oxidation of the same substrate, which is known to proceed using radical species. Accordingly, the graphene or graphite catalysis of alcohol oxidation likely proceeds via radical species.

The oxidation properties of graphene oxide or graphite oxide were studied under various conditions. In particular, in the oxidation of neat benzyl alcohol to benzylaldehyde or benzoic acid, catalyst loading was varied between 5-200 wt %, temperature was varied between 25-150° C., and reaction time was varied between 3-144 hours.

Selected reaction parameters and results are presented in Table 5. All reactions in Table 5 were allowed to proceed for 24 hours. Loading (wt %) refers to the amount of graphene oxide or graphite oxide (GO) prepared by the Hummers method added to the reaction mixture. graphene oxide or graphite oxide was prepared by the Staudenmaier method. Graphite was natural flake graphite (Bay Carbon, Inc. or Alfa Aesar). PhCHO (%) refers to the conversion of benzyl alcohol to benzylaldehyde. PhCO₂H (%) refers to the conversion of benzyl alcohol to benzoic acid. TON refers to the turnover number, which was calculated as the ratio of the moles of oxidized product to mass of graphene oxide or graphite oxide.

TABLE 5
Oxidation of benzyl alcohol to benzaldehyde
and benzoic acid under various conditions.

| | Loading | Temp. | PhCHO^{c} | PhCO₂H [symbol] | TON^{e} |
|---|---|---|---|---|---|
| Catalyst^{b} | (wt %) | (° C.) | (%) | (%) | (×10⁻²⁾ |
| GO | 20 | 100 | 24 | 0 | 1.1 |
| GO^{f} | 20 | 100 | 22 | 0 | 1.0 |
| graphite^{g} | 20 | 100 | 0 | 0 | — |
| GO | 5 | 75 | 5 | 0 | 0.93 |
| GO | 5 | 100 | 5 | 0 | 0.93 |
| GO | 5 | 150 | 6 | 0 | 1.1 |
| GO | 20 | 75 | 12 | 0 | 0.56 |
| GO | 20 | 100 | 24 | 0 | 1.1 |
| GO | 20 | 150 | 27 | 0 | 1.3 |
| GO | 50 | 75 | 30 | 0 | 0.56 |
| GO | 50 | 100 | 61 | 0.1 | 1.1 |
| GO | 50 | 150 | 85 | 12 | 1.6 |
| GO | 200 | 75 | 73 | 4 | 0.36 |
| GO | 200 | 100 | 92 | 7 | 0.43 |
| GO | 200 | 150 | 49 | 51 | 0.46 |

[symbol]

indicates data missing or illegible when filed

At temperatures less than or equal to 75° C., alcohol conversion peaked at 73 mol % even with reaction times as long as 144 hours and catalyst loading as high as 200 wt %. At temperatures above 100° C., conversion rates as high as 85 mol % were observed with loading above 50 wt %. Conversion appeared to plateau at 6 hours under these conditions, indicating decomposition of the catalyst. At temperatures above or equal to 100° C., an appreciable amount of PhCO₂H, which may not be desirable, was observed and the content of this acid increased with temperature, catalyst loading, and reaction time. Use of 200 wt % catalyst for a reaction time of 24 hours at 100° C. afforded a high conversion rate of 99% with an aldehyde:acid ratio of 92:7.

The turnover numbers in Table 5 remained relatively constant at 10⁻² mol/g regardless of the reaction conditions, indicating that the catalyst consistently reached maximal activity.

The scope and reactivity of the graphene oxide catalyst was confirmed using the reaction conditions of 200 wt % and 100° C. for 24 hours. A variety of primary and secondary benzylic and aliphatic alcohols were tested. The results of these tests are summarized in Table 6. For alcohol number 2 in the table, 2 mol % benzaldehyde was also found. For alcohol number 3, a mixture of products was observed after 24 hours at 100° C. with 200 wt % graphene oxide or graphite oxide. The reported results are for a reaction for 24 hours at 75° C. in 50 wt % graphene oxide or graphite oxide.

TABLE 6
Oxidation of various alcohols.

| En- | | | Con- |
|---|---|---|---|
| try | Alcohol | Product | version |
| 1^{b} | 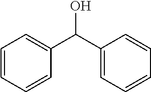 [SMILES: OC(c1ccccc1)c1ccccc1] | 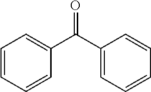 [SMILES: O=C(c1ccccc1)c1ccccc1] | 98% |
| 2^{b} | 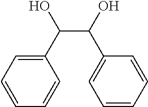 [SMILES: OC(c1ccccc1)C(O)c1ccccc1] | 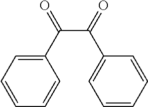 [SMILES: O=C(C(=O)c1ccccc1)c1ccccc1] | 96%^{c} |
| 3^{d} | 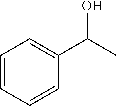 | 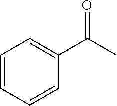 | 26% |
| 4 | 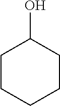 | 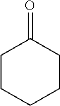 | >98% |
| 5^{d} | 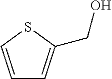 | 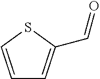 | 18% |

Overall, benzylic alcohols afforded the highest conversion, particularly dibenzylic alcohols such as benzhydrol (number 1 in Table 6). Cyclohexanol (number 4 in Table 6) showed conversion rates similar to benzyl alcohol. 2-thiophenemethanol (number 5 on Table 6) showed modest conversion rates with no sulfur oxidation, confirming the alcohol selectivity of the catalyst and its tolerance of functional groups.

### Example 8

### Hydration of Alkynes

Various alkynes were reacted with graphene oxide or graphite oxide to form their ketone hydration products as shown in Table 7. In the reactions the alkyne was reacted with 200 wt % graphene oxide or graphite oxide at 100° C. for 24 hours. In example number 4, 24 mol % of 1-(4-ethynylphenyl)ethanone was also produced.

TABLE 7
Hydration of various alkynes.

| Entry | Alkyne | Product | Conversion |
|---|---|---|---|
| 1 | 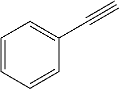 | 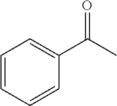 | >98% |
| 2 | 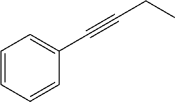 | 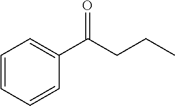 | 52% |
| 3 | 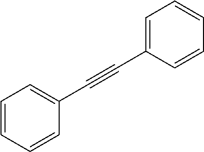 [SMILES: C(#Cc1ccccc1)c1ccccc1] | 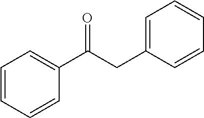 [SMILES: O=C(Cc1ccccc1)c1ccccc1] | 41% |
| 4^{b} | 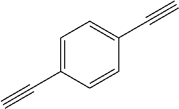 | 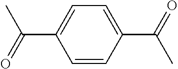 | 26% |
| 5 | C₈H₁₇—≡ | 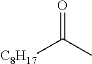 | 27% |

Graphene oxide or graphite oxide was also able to catalyze high conversion rates in this reaction even at the relatively low 100° C. temperature used. As in the alcohol oxidation reactions, aromatic alkynes exhibited higher conversion rates than their aliphatic analogues. Alkynes are known to undergo hydration under acidic conditions at temperatures above 200° C., so it is likely that graphene oxide or graphite oxide catalyzes the alkyne hydration reaction due to the acidic nature of the graphene oxide or graphite oxide.

### Example 9

### Chalcone Synthesis

In a typical preparation, a 7.5 mL vial was charged with 50-200 mg of graphene oxide or graphite oxide, methyl ketone or alkyne (0.5 mmol), aldehyde or alcohol (0.5 or 1 mmol) and a magnetic stir bar. The vial was then sealed with a Teflon-lined cap under ambient atmosphere and heated at 80-150° C. for 14-24 h. Afterward, the reaction mixture was cooled to room temperature and washed with 50 mL CH₂Cl₂. The filtrate was collected and the solvent was evaporated to obtain the crude product. Following purification of the crude product by column chromatography on silica gel, the desired product was dried under vacuum.

The acidic and hygroscopic properties inherent to graphene oxide or graphite oxide as a means to effect C—C bond-forming reactions. As summarized in Table 8, a series of coupling reactions involving various acetophenones and benzaldehydes was performed neat by combining equimolar quantities of the aforementioned substrates with a fixed quantity of graphene oxide or graphite oxide. After 14 h at 80° C., the GO was removed via vacuum filtration and the corresponding chalcone products were isolated by silica chromatography in good yields (66 to 85%). Although electron poor as well as electron rich aromatic compounds were successfully coupled, limited reactivities were observed when various aliphatic species (e.g., octanal, 2-pentanone, and hexane-2,5-dione) were explored as substrates. These results suggest that the graphene oxide or graphite oxide was not merely functioning as an exogenous acid source and that aliphatics either do not possess sufficient rigidity required for an unencumbered approach to the graphene oxide or graphite oxide surface or that a favourable arene-graphene oxide or graphite oxide interaction is needed.

TABLE 8
Coupling of acetophenones to benzaldehydes.^{a}

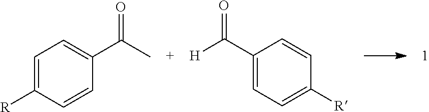

| | | | Isolated |
|---|---|---|---|
| Entry | R—C(O)CH₃ | R′—C(O)H | Yield of 1 |
| 1 | R = Ph | R′ = Ph | 81% |
| 2 | R = Ph | R′ = p-NO₂Ph | 66% |
| 3 | R = Ph | R′ = p-CH₃OPh | 76% |
| 4 | R = Ph | R′ = p-CH₃Ph | 80% |
| 6 | R = p-NO₂Ph | R′ = Ph | 85% |
| 7 | R = p-CH₃OPh | R′ = Ph | 68% |
| 8 | R = p-CH₃Ph | R′ = Ph | 74% |

^{a}All reactions were performed neat in 1:1 stoichiometry (0.5 mmol each) of ketone:aldehyde using 50 mg of graphene oxide or graphite oxide at 80° C. for 14 h.

The ability of graphene oxide or graphite oxide to facilitate oxidations (e.g., alcohols to aldehydes, a reaction shown to be catalytic in molecular oxygen) was combined with its ability to promote acid-catalyzed condensations. As shown in Table 9, in situ oxidation of benzylic alcohols to their respective benzaldehydes in combination with coupling to acetophenones was explored. Treating equimolar quantities of various alcohols and ketones with graphene oxide or graphite oxide under similar conditions as those described above afforded the expected chalcone products in isolated yields up to 55% after 24 h. depending on the substrates employed. While in situ oxidations of alcohols have been used previously in the synthesis of chalcones, metal-based catalysts were required and mixtures of products were obtained. Only a single product was observed in the crude NMR spectrum when graphene oxide or graphite oxide was used to facilitate similar tandem oxidation-condensation reactions. To confirm that the reactivities observed were not metal-mediated, inductively coupled plasma mass spectrometry was performed on an aqueous dispersion of graphene oxide or graphite oxide. The Mn content (KMnO₄ was used to prepare graphene oxide or graphite oxide) was found to be <1 ppb, similar to other native metal contaminants such as Al (3 ppb), Pb (4 ppb), and Ba (1 ppb).

TABLE 9
Coupling of acetophenones to benzyl alcohols.^{a}

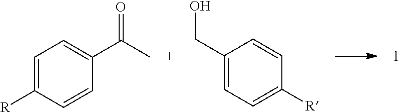

| | | | Isolated |
|---|---|---|---|
| Entry | R—C(O)CH₃ | R′—CH₂OH | Yield of 1 |
| 1 | R = Ph | R′ = Ph | 55% |
| 2 | R = Ph | R′ = p-NO₂Ph | 36%^{b} |
| 3 | R = Ph | R′ = p-CH₃OPh | ^{c} |
| 4 | R = p-NO₂Ph | R′ = Ph | 55% |
| 5 | R = p-CH₃OPh | R′ = Ph | 48% |

^{a}All reactions were performed neat in 1:1 stoichiometry (0.5 mmol each) of ketone:alcohol using 200 mg of graphene oxide or graphite oxide at 80° C. for 24 h.
^{b}Reaction performed at 100° C.
^{c} Not determined as p-methoxybenzyl alcohol was found to polymerize under these conditions.

### Example 10

### Tandem Oxidation-Condensation

Graphene oxide or graphite oxide's ability to effect hydrations of phenylacetylenes to their respective acetophenones was combined with graphene oxide or graphite oxide's ability to facilitate acid catalyzed condensations with aldehydes in a single reaction vessel, as shown in Table 10. Combination of a 1:1 molar ratio of phenylacetylene and benzaldehyde with an equimass quantity of graphene oxide or graphite oxide at 80° C. afforded the desired chalcone product in 51% isolated yield, which was increased to 79% (>98% by NMR spectroscopy) when two equiv of the aldehyde was used. It appears that some of the aldehyde was lost via oxidation to its carboxylic acid (observed via NMR spectroscopy), and that the use of excess substrate aided in driving the reaction toward formation of the targeted product. Regardless, a variety of alkyne and aldehyde derivatives were successfully coupled using this methodology.

TABLE 10
Coupling of aryl alkynes to benzaldehydes.^{a}

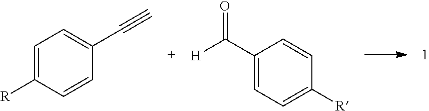

| | | | Isolated |
|---|---|---|---|
| Entry | R—C≡CH | R′—C(O)H | Yield of 1 |
| 1 | R = Ph | R′ = Ph | 51% |
| 2 | R = Ph | R′ = p-NO₂Ph | 63% |
| 3 | R = Ph | R′ = p-CH₃OPh | 43% |
| 4 | R = p-t-BuPh^{b} | R′ = Ph | 55% |
| 5 | R = p-NO₂Ph | R′ = Ph | 19%^{c} |
| 6 | R = p-CH₃OPh | R′ = Ph | 50% |

^{a}All reactions were performed neat in 1:1 stoichiometry of alkyne:aldehyde (0.5 mmol each) using 50 mg of graphene oxide or graphite oxide at 100° C. for 14 h.
^{b}Reaction of this substituted phenylacetylene derivative with benzaldehyde was used to help elucidate the coupling mechanism
^{c}The high melting point (150° C.)¹⁶ of p-nitrophenylacetylene limited miscibility, and ultimately reactivity, under the conditions explored.

The ability of graphene oxide or graphite oxide to catalyze the aforementioned hydration and oxidation reactions in a single reaction vessel, and then facilitate an acid-catalyzed Claisen-Schmidt type condensation coupling of the respective products formed in situ was also demonstrated. As shown in Table 11, the combination of various phenylacetylenes and benzyl alcohols (1:2 molar ratio) with graphene oxide or graphite oxide under conditions similar to those described above afforded the desired chalcone products in up to 61% isolated yields (94% conversion by NMR spectroscopy). This methodology may prove useful in syntheses where methyl ketones are inaccessible, or unwanted side reactions (e.g., irreversible formation of ketols or Michael addition products) are observed.

TABLE 11
Coupling of aryl alkynes to benzyl alcohols.^{a}

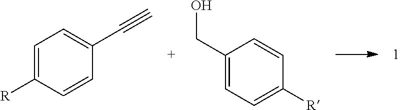

| | | | Isolated |
|---|---|---|---|
| Entry | R—C≡CH | R′—CH₂OH | Yield of 1 |
| 1 | R = Ph | R′ = Ph | 61%^{b} |
| 2 | R = Ph | R′ = p-NO₂Ph | 34%^{c} |
| 3 | R = Ph | R′ = p-CH₃OPh | ^{b, d} |
| 4 | R = p-NO₂Ph | R′ = Ph | 10%^{e} |
| 5 | R = p-CH₃OPh | R′ = Ph | 50%^{b} |

^{a}All reactions were performed neat in 1:2 stoichiometry (0.5 mmol of alkyne; 1 mmol of alcohol) using 200 mg of graphene oxide or graphite oxide at 80 or 100° C. for 24 h.
^{b}Performed at 80° C.
^{c}Performed at 100° C.
^{d} Not determined as p-methoxybenzyl alcohol was found to polymerize under these conditions.
^{e}Performed at 150° C. The high melting point (150° C.) of p-nitrophenylacetylene limited miscibility, and ultimately reactivity, under the conditions tested.

### Example 11

### Oxidation of Thiophenol

In a preliminary experiment, thiophenol (25 mg) was treated with GO (75 mg) at 100° C. in CHCl₃ (0.3 mL) for 10 min in a sealed vessel. After workup by dissolution of the crude reaction mixture in CHCl₃ (50 mL) followed by filtration of the heterogeneous carbon and removal of the volatile solvent, the thiol was determined by ¹H NMR spectroscopy (CDCl₃) to have been quantitatively converted to diphenyldisulphide. Compared to previously reported oxidative couplings of thiols using metal catalysts, significantly less time was required (10 min versus several hours) to achieve quantitative reaction using GO.

As shown in Table 12 below, minimal changes in the conversion were observed upon increasing the reaction time (entries 2-5). While 20 wt % GO afforded 81% of the target product (entry 6), increasing the loading to 60 wt % GO was found to be sufficient to drive the oxidation reaction to quantitative conversion (entry 7). We also found that variations in the reaction temperature (ranging from 40-100° C.) had a significant effect on the isolated yield of the disulphide product (entries 7-10). At low temperatures, minimal yield of product was obtained; however, a quantitative conversion was generally observed at reaction temperatures at or above 100° C. Minimal yields of the disulphide product were obtained when natural flake graphite, activated carbon, or hydrazine-reduced graphene oxide were substituted for GO (entries 11-13). Likewise, only a low yield of diphenyldisulphide (3.5%; entry 1) was obtained in the absence of GO under otherwise identical conditions (100° C., 10 min).

TABLE 12
Optimization of the GO-catalyzed oxidation of thiophenol.^{a}

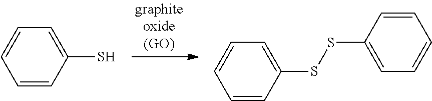 [SMILES: Sc1ccccc1 ; c1ccc(SSc2ccccc2)cc1]

| | | Reaction | | |
|---|---|---|---|---|
| | Carbon | temperature | Reaction | Conversion^{b} |
| Entry | (loading) | (° C.) | time | (%) |
| 1 | — | 100 | 10 min | 3.5 |
| 2 | GO (300 wt %) | 100 | 10 min | 100 |
| 3 | GO (300 wt %) | 100 | 20 min | 98 |
| 4 | GO (300 wt %) | 100 | 30 min | 94 |
| 5 | GO (300 wt %) | 100 | 60 min | 96 |
| 6 | GO (20 wt %) | 100 | 10 min | 81 |
| 7 | GO (60 wt %) | 100 | 10 min | 100 |
| 8 | GO (60 wt %) | 40 | 10 min | 5 |
| 9 | GO (60 wt %) | 60 | 10 min | 22 |
| 10 | GO (60 wt %) | 80 | 10 min | 86 |
| 11 | Graphite (60 wt %) | 100 | 10 min | 7 |
| 12 | Activated carbon | 100 | 10 min | 10 |
| | (60 wt %) | | | |
| 13 | Hydrazine-reduced | 100 | 10 min | 23 |
| | graphene oxide^{c} | | | |
| | (60 wt %) | | | |

^{a}Reaction conditions: thiophenol (25 mg), carbon (type and amount indicated), and CDCl₃ (0.3 mL) were combined in sealed 7.5 mL vial for the time and temperature indicated.
^{b}Conversion of thiophenol to diphenyldisulphide was monitored by ¹H NMR spectroscopy (CDCl₃) using 1,4-dinitrobenzene as an internal standard.
^{c}Prepared according to previously reported methods.

### Example 12

### Oxidation of Thiols Containing Compounds

Using the optimized experimental conditions described above (25 mg thiol, 15 mg GO, 0.3 mL CHCl₃, 100° C., 10 min), the substrate scope of the method was explored. As shown in Table 13, a wide range of thiols showed excellent conversion when treated with GO, and their pure disulphide products were obtained after short reaction times by dissolution of the crude reaction mixtures in CHCl₃ (50 mL), filtration of the residual carbon, removal of the volatile solvent under vacuum, and purification via column chromatography. The reactivity of alkyl thiols (entries 1-3) appeared slightly lower than that of aryl thiols (entries 4-10), consistent with our previous observation that GO often exhibits higher activity toward arene-functionalized substrates.^{4,6,7} As a further indicator of preferential reactivity of aryl thiols over alkyl thiols, a 1:1 molar mixture of thiophenol and 1-butanethiol (total mass: 25 mg) was treated with GO (60 wt %) under optimized conditions (100° C., 10 min). Using ¹H NMR spectroscopy, the crude product mixture (63% conversion) was found to contain a 1.0:5.0:2.9 molar ratio of dibutyldisulphide:butylphenyldisulphide:diphenyldisulphide, as well as unreacted 1-butylsulphide (34%) and a small amount of unreacted thiophenol (<5%).

Perturbation of the electronic properties of aromatic thiols through the incorporation of electron-donating or -withdrawing substituents did not significantly affect the isolated yield of the desired product. For example, both electron rich (entries 4-7) and electron deficient thiols (entries 8-10) gave excellent isolated yields of the respective disulphides (94-99%). No over-oxidation (including N-oxidation, in the case of 2-aminothiophenol) was observed in any of the reactions performed, as has been observed with chromic potassium sulphate (CrK(SO₄)₂.12H₂O) and other transition metal-based oxidation catalysts.

TABLE 13
Oxidation of various thiols using GO.^{a}

| Entry | Starting material | Product | Reaction time | Yield^{b} (%) |
|---|---|---|---|---|
| 1 | 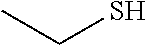 | 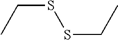 | 30 min^{c} | 77 |
| 2 | 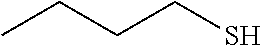 | 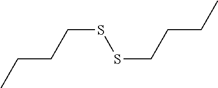 | 30 min^{c} | 90 |
| 3 | 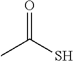 | 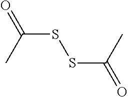 | 10 min | 92 |
| 4 | 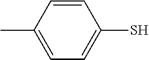 | 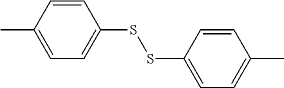 | 10 min | 95 |
| 5 | 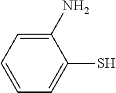 | 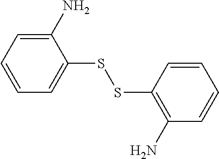 [SMILES: Nc1ccccc1SSc1ccccc1N] | 10 min | 96 |
| 6 | 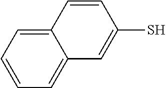 [SMILES: Sc1ccc2ccccc2c1] | 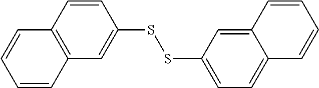 [SMILES: c1ccc2cc(SSc3ccc4ccccc4c3)ccc2c1] | 10 min | 97 |
| 7 | 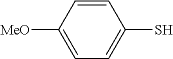 | 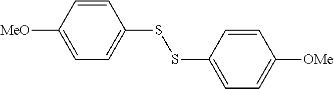 [SMILES: COc1ccc(SSc2ccc(OC)cc2)cc1] | 10 min | 97 |
| 8 | 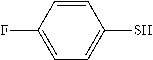 | 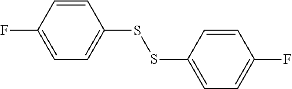 [SMILES: Fc1ccc(SSc2ccc(F)cc2)cc1] | 10 min | 99 |
| 9 | 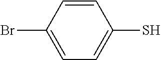 | 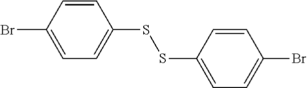 [SMILES: Brc1ccc(SSc2ccc(Br)cc2)cc1] | 10 min | 94 |
| 10 | 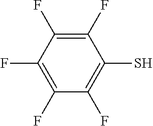 [SMILES: Fc1c(F)c(F)c(S)c(F)c1F] | 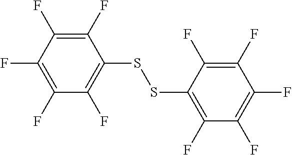 [SMILES: Fc1c(F)c(F)c(SSc2c(F)c(F)c(F)c(F)c2F)c(F)c1F] | 10 min | 98 |
| 11 | 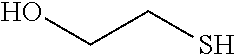 | 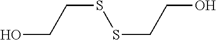 | 30 min^{c} | 75 |

^{a}All reactions were performed at 100° C. in a sealed 7.5 mL vial using 25 mg thiol, 15 mg GO (60 wt %) and 0.3 mL CHCl₃ for the indicated reaction time.
^{b}Isolated yield after purification by column chromatography.
^{c}Low yields (40-65%) were observed after 10 min; higher yields were obtained after 30 min.

### Example 13

### Oxidation of Sulfides

Diphenylsulphide was oxidized to diphenylsulphoxide in 86% isolated yield after 24 h at 100° C. (300 wt % GO) (Scheme 1); no other oxidation products were observed in the crude reaction mixture. In a similar manner to that described above, the reaction was optimized with respect to GO loading, reaction temperature, and reaction time. We found that GO loadings below 300 wt % were less effective in affording the targeted products; however, no improvement in isolated yield was observed at higher loadings. Though the loadings used herein were higher than those typically used with metal catalysts, GO's low cost and heterogeneity make it a practical choice. As observed in the aforementioned thiol coupling reactions, minimal reactivity was observed when carbons other than GO were used (i.e., graphite, activated carbon, or hydrazine-reduced graphene oxide).

As summarized in Table 14, a broad range of sulphides was successfully converted to their corresponding sulphoxides using GO under optimized conditions (25 mg sulphide, 75 mg GO, 0.3 mL CHCl₃, 100° C., 24 h). Excellent yields of the targeted products were obtained in most cases after purification by column chromatography, including those that featured S-alkyl substituents. Variations in the electronic structure of the arene-substituted sulphides had a slight influence on the yield of sulphoxide product. For example, an electron rich sulphide (entry 8) was oxidized in 96% isolated yield, whereas an electron deficient analogue (entry 9) was obtained in 81% yield under otherwise identical conditions. Compared to previously reported Ru and Fe catalysts (utilizing O₂ or H₂O₂ as the oxidant), GO provides increased yield and increased selectivity for the sulphoxide over the sulphone.

TABLE 14
Oxidation of various sulphides by graphite oxide.^{a}

| Entry | Starting material | Product | Yield^{b} |
|---|---|---|---|
| 1 | 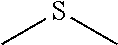 | 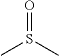 | 89% |
| 2 | 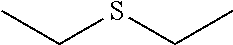 | 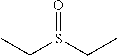 | 92% |
| 3 | 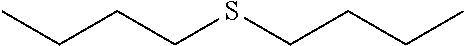 | 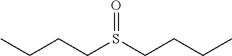 | 90% |
| 4 | 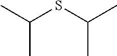 | 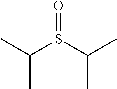 | 51% |
| 5 | 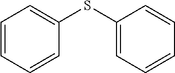 [SMILES: c1ccc(Sc2ccccc2)cc1] | 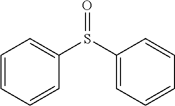 [SMILES: O=S(c1ccccc1)c1ccccc1] | 86% |
| 6 | 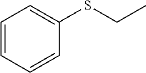 | 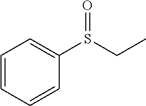 | 85% |
| 7 | 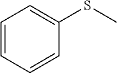 | 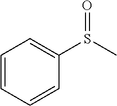 | 75% |
| 8 | 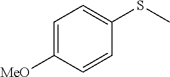 | 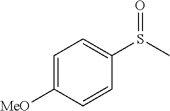 | 96% |
| 9 | 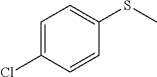 | 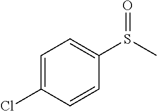 | 81% |

^{a}All reactions were performed at 100° C. in a sealed 7.5 mL vial using 25 mg sulphide, 75 mg GO (300 wt %) and 0.3 mL CHCl₃ for 24 h.
^{b}Isolated yield after purification by column chromatography.

### Example 14

### Optimization Studies

The oxidation of diphenylsulphide using GO was optimized with respect to GO loading (Table 15), reaction temperature (Table 16), and reaction time (Table 17). Additionally, to explore how dialkylsulphides behaved compared to diarylsulphides, the oxidation of dibutylsulphide was optimized with respect to GO loading (Table 18).

TABLE 15
Oxidation of diphenylsulphide to diphenysulfone
under varying GO loadings.^{a}

| Entry | GO | Conversion (%)^{b} |
|---|---|---|
| 1 | 25 mg | 32 |
| 2 | 50 mg | 51 |
| 3 | 75 mg | 89 |
| 4 | 100 mg | 90 |

^{a}All reactions were performed at 100° C. in a sealed 7.5 mL vial with 25 mg diphenylsulphide, the indicated amount of GO, and 0.3 mL CDCl₃ for 24 h. The products were extracted in CDCl₃ (1 mL) and separated by syringe filtration (0.2 μm PTFE).
^{b}The indicated conversions were determined by ¹H NMR spectroscopy via integration of appropriate non-overlapping peaks using 1,4-dinitrobenzene as an internal standard.

TABLE 16
Oxidation of diphenylsulphide to diphenysulfone
under varying reaction temperatures.^{a}

| Entry | Reaction temperture | Conversion (%)^{b} |
|---|---|---|
| 1 | 60° C. | 0 |
| 2 | 80° C. | 6 |
| 3 | 100° C. | 89 |
| 4 | 120° C. | 78 |

^{a}All reactions were performed at the indicated temperature in a sealed 7.5 mL vial with 25 mg diphenylsulphide, 75 mg (300 wt %) GO, and 0.3 mL CDCl₃ for 24 h. The products were extracted in CDCl₃ (1 mL) and separated by syringe filtration (0.2 μm PTFE).
^{b}The indicated conversions were determined by ¹H NMR spectroscopy via integration of appropriate non-overlapping peaks using 1,4-dinitrobenzene as an internal standard.

TABLE 17
Oxidation of diphenylsulphide to diphenysulfone
under varying reaction times.^{a}

| Entry | Reaction time | Conversion (%)^{b} |
|---|---|---|
| 1 | 13 h | 24 |
| 2 | 24 h | 89 |
| 3 | 37 h | 87 |

^{a}All reactions were performed at 100° C. in a sealed 7.5 mL vial with 25 mg diphenylsulphide, 75 mg (300 wt %) GO, and 0.3 mL CDCl₃ for the indicated time. The products were extracted in CDCl₃ (1 mL) and separated via syringe filtration (0.2 μm PTFE).
^{b}The indicated conversions were determined by ¹H NMR spectroscopy via integration of appropriate non-overlapping peaks using 1,4-dinitrobenzene as an internal standard.

TABLE 18
Oxidation dibutylsulphide to diphenysulfone
using various GO loadings.^{a}

| Entry | GO | Conversion (%)^{b} |
|---|---|---|
| 1 | 25 mg | 25 |
| 2 | 50 mg | 46 |
| 3 | 75 mg | 96 |

^{a}All reactions were performed at 100° C. in a sealed 7.5 mL vial with 25 mg dibutylsulphide, the indicated amount of GO, and 0.3 mL CDCl₃ for 24 h. The products were extracted in CDCl₃ (1 mL) and separated by syringe filtration (0.2 μm PTFE).
^{b}The indicated conversions were determined by ¹H NMR spectroscopy via integration of appropriate non-overlapping peaks using 1,4-dinitrobenzene as an internal standard.

### Example 15

### Spent Catalyst from Sulfur Oxidation Reaction

To ascertain the fate of the GO used in the aforementioned oxidation reactions, the residual carbon was separated from a reaction mixture containing 25 mg thiophenol, 15 mg GO, 0.3 mL CDCl₃ after 10 min at 100° C. by filtration and the resulting material was recovered by filtration, dried under vacuum and characterized.

Compared to the GO starting material, the FT-IR spectrum (KBr) of the recovered carbon exhibited attenuated stretching frequencies ascribed to the C—OH, C═O, and C—O moieties. The FT-IR spectrum also revealed new signals attributable to the presence of aromatic and olefinic species. The deoxygenation of GO was later confirmed via X-ray photoelectron spectroscopy (XPS) and elemental combustion analysis. For example, while the as-prepared GO exhibited a C:O ratio of 2.6:1 by XPS, the material recovered after reacting GO with thiophenol showed a C:O ratio of 9.2:1.

Table 19 shows Elemental Combustion Analysis performed on samples of as-prepared GO and the carbon material recovered after reacting thiophenol with GO (60 wt %) at 100° C. for 10 min. The products were dissolved in 50 mL of CHCl₃ and the residual carbon material was recovered by filtration and dried under vacuum.

TABLE 19
Summary of combustion analysis data.

| | Starting GO^{a} | Recovered Carbon^{b} |
|---|---|---|
| Carbon | 53.48 | 66.46 |
| Hydrogen | 1.80 | 1.49 |
| Nitrogen | none found | none found |
| Oxygen | 39.27 | 29.39 |
| Sulfur | 0.76 | 1.09 |
| Chlorine | none found | 1.49^{c} |
| TOTAL | 95.31 | 99.92 |

^{a}Prepared via the modified Hummers method described above.
^{b}Material recovered after heating 25 mg of thiophenol in the presence of 0.15 g (60 wt %) of GO and 0.3 mL CHCl₃ at 100° C. for 10 min, followed by dissolution of the crude mixture in 50 mL CHCl₃ and isolation of the carbon product by filtration.
^{c}The chlorine content in the recovered carbon product is believed to be due to the presence of CHCl₃ that cannot be removed.

Overall, these results demonstrate that graphene oxide or graphite oxide may catalyze a number of related reactions with little or no change in the reaction conditions.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein is employed in practicing the invention. It is intended that the following claims define the scope of the invention and that methods and structures within the scope of these claims and their equivalents be covered thereby.

1. A method for chemically transforming an organic compound, comprising:
(a) contacting the organic compound with a catalytically active carbocatalyst having a surface terminated with one or more peroxide, hydroxyl, aldehyde, or carboxylic acid groups; and
(b) transforming the organic compound with the aid of the catalytically active carbocatalyst to form a mixture of a reaction product and a spent or partially spent carbocatalyst.

2. The method of claim 1, wherein the catalytically active carbocatalyst is an oxidized form of graphite.

3. The method of claim 2, wherein the catalytically active carbocatalyst is graphene oxide or graphite oxide.

4. The method of claim 1, wherein the catalytically active carbocatalyst is an oxidized carbon-containing material.

5. The method of claim 1, wherein the catalytically active carbocatalyst is characterized by one or more FT-IR features at about 3150 cm⁻¹,1685 cm⁻¹, 1280 cm⁻¹, or 1140 cm⁻¹.

6-29. (canceled)

30. The method of claim 1, wherein the organic compound has at least one sp³-hybridized C—H bond, and the transformation involves the activation of the sp³-hybridized C—H bond by means of the graphene oxide or graphite oxide.

31-33. (canceled)

34. The method of claim 3, wherein the organic compound is brought in contact with the graphene oxide or graphite oxide catalyst for a time of at least 1 second and/or at a temperature of at least 0° C.

35. The method of claim 3, wherein the graphene oxide or graphite oxide or carbocatalyst has less than 1 part per million transition metal.

36-44. (canceled)

45. The reaction mixture formed according to the method of claim 1.

46. The reaction product formed according to the method of claim 1.

47. A method for transforming an organic compound having a least one C—H bond, the method comprising:
a. providing a catalytically active carbocatalyst exhibiting one or more Fourier Transform Infrared (FT-IR) feature at about 3150 cm⁻¹,1685 cm⁻¹, 1280 cm⁻¹, or 1140 cm⁻¹; and
b. contacting the organic compound with the catalytically active carbocatalyst, thereby transforming the C—H bond of the organic compound, and converting the carbocatalyst to a carbon-containing surface, the carbon-containing surface exhibiting an FT-IR spectrum with one or more attenuated feature at about 3150 cm⁻¹,1685 cm⁻¹, 1280 cm⁻¹, or 1140 cm⁻¹.

48. A reaction mixture for use in an oxidation, hydration, dehydrogenation or condensation reaction, comprising:
a. an organic molecule; and
b. a catalytically active carbocatalyst for facilitating the oxidation, hydration, dehydrogenation or condensation reaction of the organic molecule, the carbocatalyst having less than 1 part per million metal.

49. The reaction mixture of claim 48, wherein the carbocatalyst exhibits one or more Fourier Transform Infrared (FT-IR) feature at about 3150 cm⁻¹, 1685 cm⁻¹, 1280 cm⁻¹ or 1140 cm⁻¹.

50. The reaction mixture of claim 48, wherein the catalyst has less than 1000 wt % graphene oxide or graphite oxide.

51-84. (canceled)

85. The reaction mixture of claim 48, further comprising an additional oxidizing agent.

86-89. (canceled)
